(19) United States
(12) Patent Application Publication
(10) Pub. No.: US 2005/0170502 A1
(43) Pub. Date: Aug. 4, 2005

(54) Methods for maintaining hepatocytes in culture and for differentiating embryonic stem cells along a hepatocyte lineage

(75) Inventors: Mark A. Zern, Sacramento, CA (US); Hitoshi Shirahashi, Nakama City (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA (US)

(21) Appl. No.: 10/956,169

(22) Filed: Sep. 30, 2004

Related U.S. Application Data
(60) Provisional application No. 60/507,786, filed on Sep. 30, 2003.

(51) Int. Cl. C12N 5/08

(52) U.S. Cl. 435/370

(57) ABSTRACT

The invention provides methods and media for culturing embryonic stem (ES) cells, such as human ES cells, and directing them along the hepatic lineage. It further provides methods for maintaining hepatocytes in culture for extended periods. The invention further provides cells cultured by the methods of the invention. Additionally, the invention provides methods of transducing cells with marker proteins that will be expressed only in hepatocyte-like cells and selecting for cells expressing the marker protein.

### CROSS-REFERENCES TO RELATED APPLICATIONS

This application claims priority from U.S. Provisional Application 60/507,786, filed Sep. 30, 2003, the contents of which are incorporated herein for all purposes.

### STATEMENT AS TO RIGHTS TO INVENTIONS MADE UNDER FEDERALLY SPONSORED RESEARCH AND DEVELOPMENT

This invention was made in part with government support under grant AA-06386 awarded by the National Institute of Alcohol Abuse and Alcoholism of the National Institutes of Health. The government has certain rights in the invention.

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

Liver transplantation is the only established successful treatment for end-stage liver diseases; however, the number of donor livers is inadequate. As alternatives, extracorporeal bioartificial liver support devices (Allen, J. W. et al., Hepatology 34:447-455 (2001)) and hepatocyte transplantation (Fox, I. J. et al., N. Engl. J. Med. 338:1422-1426 (1998); Strom, S. C. et al., Transplantation 63:559-569 (1997)) offer the possibility of effective treatment for many inherited and acquired hepatic disorders, including fulminant hepatic failure, end-stage cirrhosis, and liver-based congenital metabolic disease. Unfortunately, the lack of donor livers makes it difficult to obtain enough viable human hepatocytes for the further advancement of hepatocyte-based therapies (Kobayashi, N. et al., Transplantation 69:202-207 (2000); Nakamura, J. et al., Transplantation 63:1541-1547 (1997)). Therefore, currently available bioartificial liver devices employ tumor-derived cell lines or animal cells. Both approaches have potential problems such as possible tumor cell seeding, pathologic immune responses, and xenozoonoses. Thus, it would be advantageous if functional human hepatocytes could be gene rated from other sources. Embryonic stem (ES) cells (Hamazaki, T. et al., FEBS. Lett. 497:15-19 (2001)), bone marrow stem cells (Schwartz, R. E. et al., J. Clin. Invest. 109:1291-1302 (2002)), and liver stem cells (oval cells) (Overturf, K. et al., Am. J. Pathol. 151:1273-1280 (1997)), have been considered candidate cell types with potential to develop into viable hepatocytes. Conditions and methods for directing stem cells to differentiate into a specific lineage, such as hepatocytes, however, have not yet been determined.

ES cells are continuously growing stem cell lines of embryonic origin first isolated from the inner cell mass of blastocysts from the developing embryo (Evans, M. J. et al., Nature 292:154-156 (1981); Martin, G. R. Proc. Natl. Acad. Sci. USA. 78:7634-7638 (1981)). These cells are capable of self-renewal and differentiation, and thus can theoretically provide a limitless supply of differentiated cells. Recent studies have demonstrated the plasticity of adult stem cells (Lagasse, E. et al., Nat. Med. 6:1229-1234 (2000); Petersen, B. E. et al., Science 284:1168-1170 (1999); Theise, N. D. et al., Hepatology 31:235-240 (2000)), and that mouse ES cells are able to undergo an early endodermal differentiation in vitro (Abe, K. et al., Exp. Cell. Res. 229:27-34 (1996); Barbacci, E. et al., Development 126:4795-4805 (1999)), and in vivo (Coffinier, C. et al., Development 126:4785-4794 (1999); Morrisey, E. E. et al., Genes. Dev. 12:3579-3590 (1998)). Mouse ES cell-derived cardiomyocytes (Klug, M. G. et al., J. Clin. Invest. 98:216-224 (1996); Min, J. Y. et al., J. Appl. Physiol. 192:288-296 (2002)), neural precursors (Andressen, C. et al., Stem Cells 19:419-424 (2001); Brustle, O. et al., Science 285:754-756 (1999); Brustle, O. et al., Proc. Natl. Acad Sci. USA. 94:14809-14814 (1997); Rolletschek, A. et al., Mech. Dev. 105:93-104 (2001)) and hematopoietic precursors (Potocnik, A. J. et al., Proc. Natl. Acad. Sci. USA. 94:10295-10300 (1997)) have been transplanted into recipient animals. For the differentiation of mouse ES cells, individual or a combination of growth and differentiation factors were employed in previous investigations. For example, differentiation of mouse ES cell-derived dopaminergic neurons was enhanced by interleukin-1β glial neurotrophic factor, neuroturin, transforming growth factor-β3 and dibutyryl-cyclic AMP (Rolletschek, A. et al., Mech. Dev. 105:93-104 (2001)). However, it remains unclear whether ES cells have the ability to differentiate into mature endodermal phenotypes, such as hepatocytes, in vitro. Whereas recent studies have shown that mouse ES cells could be differentiated into albumin-positive cells in vitro (Abe, K. et al., Exp. Cell. Res. 229:27-34 (1996); Hamazaki, T. et al., FEBS. Lett. 497:15-19 (2001); Jones, E. A. et al., Exp. Cell. Res. 272:15-22 (2002); Yamada, T. et al., Stem Cells 20:146-154 (2002)), and in vivo (Choi, D. et al., Cell Transplant. 11:359-368 (2002)), there has been no report of the identification and isolation of definitive hepatocytes from differentiated mouse ES cell cultures. In addition, although these studies have shown albumin gene expression in mouse ES cells, they have not evaluated the level of albumin gene expression under different culture conditions by quantitative methods (Miyashita, H. et al., Cell Transplant. 11:429-434 (2002)). Human ES cells have also been shown to differentiate into a variety of cells, such as neural precursors (Carpenter, M. K. et al., Exp. Neurol. 172:383-397 (2001)), hematopoietic precursors (Odorico, J. S. et al., Stem Cells 19:193-204 (2001)), bone tissue, and muscle cells (Michal, A., J. Anat. 200:225-232 (2002)); and the first report of human ES cells expressing a hepatocyte phenotype has recently been published (Rambhatla, L. et al., Cell Transplant. 12:1-11 (2003)).

Primary rodent hepatocytes cultures are valuable entities for a variety of basic science studies, including toxicology and pharmacology experiments. Unfortunately, hepatocytes readily dedifferentiate in cultures. Attempts to inhibit this dedifferentiation process have been undertaken for several decades, and a host of additions to standard media have been proposed, such as amino acids, vitamins, trace metals, bicarbonate, and nicotinamide, among others (Bissell, D. M. et al., J. Clin. Invest. 79:801-812 (1987); Block, G. D. et al., J. Cell. Biol. 132:1133-1149 (1996); Mitaka, T. Int. J. Exp. Pathol 79:393-409 (1998); Rogler, L. E. Am. J. Pathol. 150:591-602 (1997)). However, despite these advances, no single set of conditions which maintains differentiation over a period of time have been found to be satisfactory.

It would be useful to develop culture condition that direct ES cells along a hepatocyte lineage. It would further be useful to develop culture condition that maintain high levels of hepatocyte-specific function in long-term cultures of primary hepatocytes. The present invention fills these and other needs.

### BRIEF SUMMARY OF THE INVENTION

This invention provides cells differentiated from an embryonic stem (ES) cell along a hepatocyte lineage by culturing said ES cell in a medium with growth factors consisting essentially of insulin and dexamethasone. The insulin is preferably present in said medium in a concentration of from 0.010 U/mL to 1.5 U/mL, and more preferably present in the medium at about 0.050 to about 0.075 U/mL. The insulin in the medium is preferably human insulin. The dexamethasone is preferably present in the medium in a concentration of from 15 nM to 150 nM, and more preferably present in a concentration of from 40 nM to 60 nM. The ES cell is preferably a human ES cell. The differentiated cell preferably expresses a hepatocyte-specific protein selected from the group consisting of albumin, pre-albumin, glucose-6-phosphatase, and α1-antitrypsin. The medium further preferably comprises between about 15% to about 30% fetal bovine serum (FBS), and preferably comprises 20% FBS. In some embodiments, the medium is Iscove's modified Dulbecco's medium (IMDM). The ES cell is preferably cultured on an extracellular matrix of collagen type 1. In some embodiments, the medium further comprises sodium butyrate. The sodium butyrate is preferably present in a concentration between 0.25 mM and about 10 mM. In some embodiments, the medium further comprises dimethyl sulfoxide (“DMSO”). The DMSO is preferably present in a concentration between 0.1% and about 10%. In some embodiments, the medium further comprises both sodium butyrate and dimethyl sulfoxide.

In another group of embodiments, the invention provides isolated hepatocytes maintained in culture by culturing the hepatocytes in a medium with growth factors consisting essentially of insulin and dexamethasone. The insulin is preferably present in said medium in a concentration of from 0.010 U/mL to 1.5 U/mL, and more preferably present in the medium at about 0.050 to about 0.075 U/mL. The insulin in the medium is preferably human insulin. The dexamethasone is preferably present in the medium in a concentration of from 15 nM to 150 nM, and more preferably present in a concentration of from 40 nM to 60 nM. The medium further preferably comprises between about 15% to about 30% fetal bovine serum (FBS), and preferably comprises 20% FBS. In some embodiments, the medium is Iscove's modified Dulbecco's medium (IMDM). In some embodiments, the hepatocytes are human hepatocytes. The hepatocytes are preferably cultured on an extracellular matrix of collagen type 1. In some embodiments, the medium further comprises sodium butyrate. The sodium butyrate is preferably present in a concentration between 0.25 mM and about 10 mM. In some embodiments, the medium further comprises dimethyl sulfoxide (“DMSO”). The DMSO is preferably present in a concentration between 0.1% and about 10%. In some embodiments, the medium further comprises both sodium butyrate and dimethyl sulfoxide.

In another group of embodiments, the invention provides methods of differentiating embryonic stem (ES) cells along a hepatocyte lineage. The method comprise culturing said ES cell in a medium with growth factors consisting essentially of insulin and dexamethasone. The insulin is preferably present in said medium in a concentration of from 0.010 U/mL to 1.5 U/mL, and more preferably present in the medium at about 0.050 to about 0.075 U/mL. The insulin in the medium is preferably human insulin. The dexamethasone is preferably present in the medium in a concentration of from 15 nM to 150 nM, and more preferably present in a concentration of from 40 nM to 60 nM. The ES cell is preferably a human ES cell. The differentiated cell preferably expresses a hepatocyte-specific protein selected from the group consisting of albumin, pre-albumin, glucose-6-phosphatase, and α1-antitrypsin. The medium further preferably comprises between about 15% to about 30% fetal bovine serum (FBS), and preferably comprises 20% FBS. In some embodiments, the medium is Iscove's modified Dulbecco's medium (IMDM). The ES cell is preferably cultured on an extracellular matrix of collagen type 1. In some embodiments, the medium further comprises sodium butyrate. The sodium butyrate is preferably present in a concentration between 0.25 mM and about 10 mM. In some embodiments, the medium further comprises dimethyl sulfoxide (“DMSO”). The DMSO is preferably present in a concentration between 0.1% and about 10%. In some embodiments, the medium further comprises both sodium butyrate and dimethyl sulfoxide.

In yet another group of embodiments, the invention provides methods of maintaining a hepatocyte in culture for an extended period. The methods comprise culturing the hepatocyte cell in a medium in which growth factors consist essentially of insulin and dexamethasone. The insulin is preferably present in said medium in a concentration of from 0.010 U/mL to 1.5 U/mL, and more preferably present in the medium at about 0.050 to about 0.075 U/mL. The insulin in the medium is preferably human insulin. The dexamethasone is preferably present in the medium in a concentration of from 15 nM to 150 nM, and more preferably present in a concentration of from 40 nM to 60 nM. The hepatocyte is preferably a human hepatocyte. The medium further preferably comprises between about 15% to about 30% fetal bovine serum (FBS), and preferably comprises 20% FBS. In some embodiments, the medium is Iscove's modified Dulbecco's medium (IMDM). The ES cell is preferably cultured on an extracellular matrix of collagen type 1. In some embodiments, the medium further comprises sodium butyrate. The sodium butyrate is preferably present in a concentration between 0.25 mM and about 10 mM. In some embodiments, the medium further comprises dimethyl sulfoxide (“DMSO”). The DMSO is preferably present in a concentration between 0.1% and about 10%. In some embodiments, the medium further comprises both sodium butyrate and dimethyl sulfoxide.

In still another group of embodiments, the invention provides methods of screening a compound for its effects on a hepatocyte or on a hepatocyte activity. The methods comprise (a) contacting the compound to a cell selected from the group consisting of (i) an embryonic stem (ES) cell differentiated along a hepatocyte lineage by culturing said ES cell with a culture medium containing growth factors, wherein said growth factors consist essentially of insulin and dexamethasone, and (ii) an isolated hepatocyte maintained in culture in a culture medium containing growth factors, wherein said growth factors consist essentially of insulin and dexamethasone; (b) determining any change to the cells of step (a) contacted with said compound or in an activity of said cells of step (a) contacted with said compound; and (c) correlating the change of step (b) with the effect of the compound on a cell of step (a) or on an activity of said cell. The medium is preferably Iscove's modified Dulbecco's medium (IMDM). The insulin is preferably present in the medium at about 0.050 to about 0.075 U/mL. The insulin in the medium is preferably human insulin. The dexamethasone is preferably present in the medium in a concentration of from 40 nM to 60 nM The cell in step (a) can be selected from the group consisting of a human ES cell and a human hepatocyte cell. The medium preferably further comprises 20% FBS. The cell of step (a) is preferably cultured on an extracellular matrix of collagen type 1. The medium of step (a) can further comprise sodium butyrate. The said sodium butyrate is preferably present in a concentration between 0.25 mM and about 10 mM. The medium of step (a) can further comprise dimethyl sulfoxide (“DMSO”). The DMSO is preferably present in a concentration between 0.1% and about 10%. The medium preferably further comprises both sodium butyrate and dimethyl sulfoxide.

In yet another group of embodiments, the invention provides cell cultures for producing one or more liver proteins. The cell culture is selected from the group consisting of (i) an embryonic stem (ES) cell differentiated along a hepatocyte lineage by culturing said ES cell with a culture medium containing growth factors, wherein said growth factors consist essentially of insulin and dexamethasone, (ii) an isolated hepatocyte maintained in culture in a culture medium containing growth factors, wherein said growth factors consist essentially of insulin and dexamethasone, and (iii) a combination of cells of (a) and (b). The medium is preferably Iscove's modified Dulbecco's medium (IMDM). The insulin is preferably present in the medium at about 0.050 to about 0.075 U/mL. The insulin in the medium is preferably human insulin. The dexamethasone is preferably present in the medium in a concentration of from 40 nM to 60 nM The cell in step (a) can be selected from the group consisting of a human ES cell and a human hepatocyte cell. The medium preferably further comprises 20% FBS. The cell of step (a) is preferably cultured on an extracellular matrix of collagen type 1. The medium of step (a) can further comprise sodium butyrate. The said sodium butyrate is preferably present in a concentration between 0.25 mM and about 10 mM. The medium of step (a) can further comprise dimethyl sulfoxide (“DMSO”). The DMSO is preferably present in a concentration between 0.1% and about 10%. The medium preferably further comprises both sodium butyrate and dimethyl sulfoxide.

In yet another group of embodiments, the invention provides methods of producing a liver protein, comprising (a) providing a cell culture selected from the group consisting of (i) a culture of embryonic stem (ES) cells differentiated along a hepatocyte lineage by culturing said ES cells with a culture medium containing growth factors, wherein said growth factors consist essentially of insulin and dexamethasone, (ii) isolated hepatocytes maintained in culture in a culture medium containing growth factors, wherein said growth factors consist essentially of insulin and dexamethasone, and (iii) a combination of cells of (i) and (ii); and (b) isolating the liver protein from said culture. The medium is preferably Iscove's modified Dulbecco's medium (IMDM). The insulin is preferably present in the medium at about 0.050 to about 0.075 U/mL. The insulin in the medium is preferably human insulin. The dexamethasone is preferably present in the medium in a concentration of from 40 nM to 60 nM The cell in step (a) can be selected from the group consisting of a human ES cell and a human hepatocyte cell. The medium preferably further comprises 20% FBS. The cell of step (a) is preferably cultured on an extracellular matrix of collagen type 1. The medium of step (a) can further comprise sodium butyrate. The said sodium butyrate is preferably present in a concentration between 0.25 mM and about 10 mM. The medium of step (a) can further comprise dimethyl sulfoxide (“DMSO”). The DMSO is preferably present in a concentration between 0.1% and about 10%. The medium preferably further comprises both sodium butyrate and dimethyl sulfoxide.

The invention further provides methods for identifying cells expressing a hepatocyte-like phenotype. The methods comprise transducing a population of cells with a lentiviral vector comprising a gene encoding a marker protein, wherein the gene is operably linked to a promoter for proteins exclusively or preferentially expressed in hepatocytes, and identifying cells expressing the marker protein. The marker protein can be, for example, green fluorescent protein, red fluorescent protein, or an antibiotic. The methods further include selecting cells expressing said marker protein by fluorescence activated cell sorting.

### BRIEF DESCRIPTION OF THE DRAWINGS

FIGS. 1A and B. In vitro differentiation of ES cells. A. The in vitro differentiation protocols for mouse ES cells (A) and human ES cells (B) are illustrated. The first day of the initial differentiation was designated as day 0, and the day for the placement of EBs on coated wells was designated as day 5 for mouse ES cells and day 6 for human ES cells.

FIGS. 2A and B. Albumin gene expression in differentiated mouse ES cells treated with different growth factors. Mouse ES cells were cultured in IMDM on collagen type I pre-coated 6-well plates. RNA was extracted from cells after 33 days of culture. Albumin expression was examined by real-time quantitative RT-PCR and normalized to the expression of β-actin as endogenous control gene. Normalized expression levels were compared to ES cells cultured for 15 days on 0.1% gelatin pre-coated plates in medium without growth and differentiation factors (relative expression level=1). The control levels were ES cells cultured for 33 days on collagen type I pre-coated without growth and differentiation factors. A. Albumin expression in differentiated mouse ES cells treated with individual growth and differentiation factors. B. Albumin expression in differentiated mouse ES cells treated with growth and differentiation factors combinations. HGF=hepatocyte growth factor; hEGF=human epidermal growth factor; mEGF=mouse epidermal growth factor; OnM=oncostatin M; bFGF=basic fibroblast growth factor; aFGF=acidic fibroblast growth factor; NGF=nerve growth factor; RA=all-trans-retinoic acid; BI=bovine insulin; HI=human insulin; DXM=dexamethasone; IMDM=Iscove's modified Dulbecco's medium.

FIG. 3. Albumin gene expression in differentiated mouse ES cells cultured on different substrata pre-coatings. Mouse ES cells were cultured in DMEM with human insulin and dexamethasone. RNA was extracted on day 15. Albumin expression was examined by real-time quantitative RT-PCR, normalized to β-actin, and compared to ES cells cultured for 15 days on 0.1% gelatin pre-coated plates in medium without growth and differentiation factors (relative expression level=1). Adult mouse liver was employed as a positive control.

FIG. 4. Albumin gene expression in differentiated mouse ES cells using different culture media. Mouse ES cells were cultured with or without growth and differentiation factors using different media on collagen type I pre-coated culture wells. RNA was extracted on day 33. Albumin gene expression was examined by real-time quantitative RT-PCR, normalized to β-actin, and compared to ES cells cultured for 15 days on 0.1% gelatin pre-coated plates in medium without growth and differentiation factors (relative expression level=1). Adult mouse liver was used as a positive control. DMEM=Dulbecco's modified Eagle medium; WME=Williams' medium E; IMDM=Iscove's modified Dulbecco's medium, MAPC medium=multipotent adult progenitor cell differentiation medium; 10% FBS=10% fetal bovine serum, 20% FBS=20% fetal bovine serum. HI=human insulin; DXM=dexamethasone.

FIG. 5. Time course of albumin expression in differentiated mouse ES cells in our optimal culture condition. The optimal culture condition included IMDM, human insulin and dexamethasone supplementation and collagen type I pre-coating. RNA was extracted and gene expression levels were determined with real-time quantitative RT-PCR at multiple time points during culture in our optimal condition and standard medium (DMEM with 10% FBS). Expression levels were normalized to β-actin and compared to ES cells cultured for 15 days on 0.1% gelatin pre-coated plates in medium without growth and differentiation factors (relative expression level=1). Adult mouse liver served as a positive control. DMEM=Dulbecco's modified Eagle medium; IMDM=Iscove's modified Dulbecco's medium; HI: human insulin; DXM=dexamethasone.

FIGS. 6A to 6D. Time course of prealbumin, G6P, CK19, and GGT expression in differentiated mouse ES cells in our optimal culture condition. RNA was extracted at multiple time points during optimal culture and standard culture (DMEM with 10% FBS). Expression of various markers was determined by real-time quantitative RT-PCR. A. Time course of prealbumin expression. B. Time course of glucose-6 phosphatase (G6P) expression. C. Time course of cytokeratin 19 (CK19) expression. D. Time course of γ-glutamyl transferase (GGT) expression. Prealbumin, CK19, and GGT expression were compared to ES cells cultured for 15 days on 0.1% gelatin pre-coating in DMEM with 10% FBS (relative expression level=1). G6P expression was compared to ES cells cultured for 10 days on collagen type I pre-coating in IMDM with 10% FBS (relative expression level=1). DMEM=Dulbecco's modified Eagle medium; IMDM=Iscove's modified Dulbecco's medium; HI: human insulin; DXM=dexamethasone.

FIGS. 7A, 7B, 7B1, and 7B2. Determination of albumin synthesis in differentiated mouse ES cells by Western blot analysis (A) and immunocytochemistry (B). A. Albumin production in ES cells cultured in our optimal condition was detected by Western blot analysis at day 0-75. Thirty μg of protein extracted from ES cells and EBs, and 1 μg from adult mouse liver tissue were analyzed. Membranes were also probed for actin as housekeeping control to ensure equal loading of lanes. Undifferentiated=undifferentiated ES cells cultured on STO fibroblast feeder layers and in medium supplemented with LIF. B. Immunocytochemistry for albumin in differentiated ES cells. Fixed ES cells were incubated with primary rabbit anti-mouse albumin antibody, and then with secondary anti-rabbit IgG-fluorescein isothiocyanate conjugates. B1. On day 26 of differentiation culture, albumin-positive ES cells are shown using a fluorescence microscope (original magnification ×200). B2. Light microscopy of the same field as B1 (original magnification ×200).

FIG. 8. Urea synthesis of differentiated ES cells in comparison to primary mouse hepatocytes. Urea synthesis of ES cells cultured in our optimal condition for 7, 15, and 23 days was examined. Values are the means±SEM of 3 experiments. Urea synthesis was determined based on standards with different urea concentrations and normalized to DNA content per well. Urea levels are depicted relative to urea production measured in primary mouse hepatocytes.

FIGS. 9A and B. Time course of hepatocyte gene expression in differentiated human ES cells in our optimal culture condition. The optimal culture condition was a combination of IMDM, human insulin and dexamethasone supplementation and collagen type I pre-coating. RNA was extracted and gene expression levels were determined with real-time quantitative RT-PCR at multiple time points during culture in our optimal condition. Expression levels were normalized to GAPDH and compared to ES cells cultured for 17 days in the optimal culture condition (relative expression level=1). Primary human hepatocytes served as a positive control. A. Time course of albumin gene expression. B. Time course of α1-antitrypsin (α1-AT) gene expression. IMDM=Iscove's modified Dulbecco's medium; HI: human insulin; DXM=dexamethasone, P=human hepatocytes.

FIG. 10. Determination of albumin synthesis in differentiated human ES cells by Western blot analysis and immunocytochemistry. Albumin production in human ES cells cultured in the optimal condition was detected by Western blot analysis (A) and immunocytochemistry (B). A. Thirty μg of protein extracted from ES cells from various time points of differentiation culture and EBs, and 1 μg from the human hepatoma cell line, Hep G2, were loaded. Membranes were also probed for actin as housekeeping control to ensure equal loading of lanes. Undifferentiated=undifferentiated ES cells cultured on STO fibroblast feeder layers. B. Human ES cells at day 47 of differentiation culture in the optimal condition on a chamber slide. B1. Human albumin-positive cells were shown in clumps or as individual cells (original magnification ×200). B2. A plain image of the same field as B1 (original magnification ×200).

FIG. 11. Urea synthesis of differentiated human ES cells. Urea synthesis of human ES cells cultured in the optimal condition for 12, 31, and 43 days was examined. Values were expressed as means±SEM of 3 experiments. Urea synthesis was determined based on standards with different urea concentrations and normalized to total DNA content in each well.

FIG. 12. Hepatocyte gene expression in cultured primary mouse hepatocytes using different media. Primary mouse hepatocytes were cultured with or without growth and differentiation factors using different media on collagen type I pre-coated culture wells. RNA was extracted on day 1, 14 and 35. Hepatocyte gene expression was examined by real-time quantitative RT-PCR, normalized by β-actin, and compared to primary hepatocytes one day after isolation (relative expression level=100%). DMEM=Dulbecco's modified Eagle medium; WME=Williams'medium E; IMDM=Iscove's modified Dulbecco's medium; MAPC medium=multipotent adult progenitor cell differentiation medium; HBM=hepatoblast medium; HGM=hepatocyte growth medium; 10% FBS=10% fetal bovine serum, 20% FBS=20% fetal bovine serum. HI=human insulin; DXM=dexamethasone.

FIG. 13. Cultured primary mouse hepatocytes using different media and time in culture. A. Day 1 of primary mouse hepatocyte culture (original magnification ×200). B. Day 35 of primary mouse hepatocyte culture in optimal culture condition (original magnification ×200). C. Day 35 of culture in IMDM without human insulin and dexamethasone, few hepatocytes can be visualized (original magnification ×200). IMDM=Iscove's modified Dulbecco's medium.

### DETAILED DESCRIPTION

### INTRODUCTION

Culture conditions have been discovered that permit the differentiation of mammalian embryonic stem (ES) cells along a hepatocyte lineage and induce the differentiated ES cells to express hepatocyte-specific genes at the highest levels yet recorded for in vitro culture of cells in which differentiation has been induced. The invention has several surprising aspects. Among them are the discovery that the medium to induce and support the differentiation can be relatively minimal, and does not require factors previously reported in the art to be required. Further, it has been found in the art that the culture requirements for cells from different species or types of mammals differ, yet contrary to this expectation, the present invention finds that the same culture conditions work to induce differentiation of ES cells into cells expressing hepatocyte-specific proteins in cells from organisms as different as mice and humans. Even more surprisingly, the same relatively minimal culture conditions found to induce and support the differentiation of ES cells into cells expressing hepatocyte-specific proteins can be used to maintain mammalian hepatocytes in culture for extended periods. The invention therefore marks a major advance in areas that have frustrated researchers for years.

In view of the results obtained in cells of species as widely separated as mice and humans in the studies reported herein, it is expected that the results will be applicable to mammalian ES cells and mammalian hepatocytes in general. In some preferred embodiments, the mammalian cells cultured or maintained are murine or primate hepatocytes. In many of the uses of the invention, such as toxicology studies, it is particularly desirable to differentiate human ES cells or to maintain human hepatocytes in culture.

The studies underlying the present disclosure further show that ES cells can be differentiated into cells that express hepatocyte-specific proteins and then transduced with liver-specific lentivirus to express a protein encoded by the lentivirus.

A. Culture Conditions for Differentiating ES Cells Into Cells Expressing Hepatocyte-Specific Proteins

The studies reported herein establish that the combination of insulin and dexamethasome is very effective in promoting expression of albumin and other hepatocyte-specific proteins, including α-1 antitrypsin, pre-albumin, and glucose-6-phosphatase, at levels significantly higher than those achieved by prior methods known in the art. The studies herein therefore contradict a recent report by Chinzei et al. (Hepatology 36:22-29 (2002)) that the presence of absence of growth and differentiation factors in the presence of serum supplementation did not change albumin production in ES cells. While insulin can be used as a growth factor, the studies reported herein indicate that human insulin results in higher production of hepatocyte-specific proteins than is bovine insulin, even in the differentiation of non-primate ES cells, such as murine ES cells. Thus, while insulin is a preferred growth factor for differentiating ES cells along a hepatocyte lineage or of maintaining hepatocytes in culture, human insulin is particularly preferred.

The amount of insulin used can range from 0.001 to 2.000 U/mL, more preferably, 0.010 to about 1.5 U/mL, even more preferably about 0.020 to about 1.00 U/mL, more preferably 0.30 to about 0.90 U/mL, still more preferably about 0.40 to about 0.80 U/mL and still more preferably 0.050 to 0.075 U/mL. (In the discussion of ranges in this paragraph, the term “about” is intended to encompass a concentration that ranges down to about halfway to the next lower concentration, on the one hand, and about halfway to the next higher concentration, on the other.) In some of the studies reported herein, excellent results were found with 0.063 U/mL, which is particularly preferred.

Dexamethasone is a synthetic adrenocortical steroid designated as 9-fluoro-11β,17,21-trihydroxy-16α-methylpregna-1,4-diene-3,20-dione. The empirical formula is C₂₂H₂₉FO₅. The drug is available commercially under a host of trade names. Dexamethasone suppresses the immune response and is used clinically topically and systemically in the treatment of chronic inflammatory diseases and severe allergies. The concentrations of dexamethasone that can be used in the methods and compositions of the invention range from 1 nM to about 200 nM, preferably from about 5 nM to about 175 nM, more preferably from about 15 nM to about 150 nM, even more preferably from about 25 nM to about 100 nM, can be 30 nM to about 80 nM, more preferably about 40 to about 70 nM, even more preferably about 40 to about 60 nM, and most preferably about 50 nM, with 50 nM being the most preferred. (In the discussion of ranges in this paragraph, the term “about” is intended to encompass a concentration that ranges down to about halfway to the next lower concentration, on the one hand, and about halfway to the next higher concentration, on the other.)

Notably, except as noted further in this section, when other growth and differentiation factors were added to the culture medium, the levels of albumin gene expression were either not affected or were decreased. Thus, some of the methods of the invention permit reducing the cost of differentiating ES cells along a hepatocyte lineage or of maintaining hepatocytes in culture by removing the need to introduce growth or differentiation factors other than human insulin and dexamethasone to the culture medium. In these embodiments, it is preferable that growth and differentiation factors other than human insulin and dexamethasone are either not present or are not present in quantities that reduce the expression of hepatocyte-specific proteins, such as albumin. Whether or not any given quantity of any particular factor reduces the expression of a hepatocyte-specific protein, such as albumin, can be readily tested by assays such as Northern blots, Western blots, or, preferably, by real time quantitative RT-PCR. Exemplar assays for such measurements can also be found in the Examples, infra.

The present work also establishes that the medium makes a significant difference in promoting the expression of hepatocyte-specific genes. Iscove's modified Dulbecco's medium (IMDM) is commercially available from a number of suppliers, including HyClone (Logan, Utah), Cambrex Corp. (E. Rutherford, N.J.), JRH Biosciences (Lenexa, Kans.), and Invitrogen (Carlsbad, Calif.). It is a nutrient blend of amino acids, vitamins, carbohydrates, organic and inorganic supplements and salts and supports the culture of a wide spectrum of mammalian cell types. Optionally, IMDM may be provided with the zwitterion HEPES for extra buffering capacity, and may come without L-glutamine, or with a derivatized glutamine, to reduce ammonia formation during culturing. As noted, IMDM comprises a host of inorganic salts, amino acids, vitamins, and other components at specific concentrations which have been found to support growth of a wide spectrum of mammalian cells. Persons of skill will recognize that the precise amounts of one or more of the components might be reduced or increased to result in a medium which has the same ability to support culturing of an ES cell or of a primary hepatocyte as normal IMDM, but which might perhaps not technically be considered IMDM because the values of the altered components does not fall within the suggested range for IMDM. Such altered media based on IMDM are considered to be the equivalent of IMDM for the purposes of the invention so long as ES cells or primary hepatocytes cultured in the altered media have at least 75% of the production of hepatocyte-specific proteins as do like cells cultured in IMDM, more preferably 80%, still more preferably 85%, even more preferably 90%, and most preferably 95% or higher.

ES cells in IMDM with supplementation with fetal bovine serum (FBS) showed much higher levels of albumin expression compared to like cells cultured in either Dulbecco's modified Eagle's medium (DMEM) or in Williams' medium E (WME). The studies reported herein indicate that hepatocyte-specific protein expression drops by an order of magnitude at 10% FBS. Accordingly, FBS supplementation is preferably about 15% to about 30%. To reduce cost and the like, in practice, supplementation with more than 20% FBS is not common. Thus, it is preferred if FBS supplementation is about 20%, and 20% FBS is particularly preferred.

For better induction of differentiation, the cells can be placed on a material that provides an extracellular matrix. We have found approximately an order of magnitude better production placing the cells on collagen type I, rather than collagen type IV, fibronectin or poly-D-lysine precoated tissue culture plates. Feeder cells are not required in the cell cultures of the invention, although they may be used to expand the population of ES cells prior to initiating differentiation.

Thus, by careful manipulation of medium, sub-stratum pre-coating, FBS supplementation, and supplementation of growth and differentiation factors, hepatocyte-specific differentiation and expression of hepatocyte-specific genes can be maximized. Moreover, the culture conditions defined in the studies herein inhibited the differentiation of ES cells into other cell fates by some 30 times.

The culture conditions defined herein increase albumin expression approximately 1000 times higher than that induced by standard culture conditions. Albumin, a protein produced by the liver, is the most prevalent protein in the blood, is a major transporter of divalent cations, such as Ca⁺², and is important in maintaining the osmotic pressure of the blood, thereby keeping the fluid component of the blood from leaking out into the tissues. Albumin protein levels in mouse ES cells differentiated using the optimal culture conditions of the invention were as high as 7% of the level of adult mouse liver hepatocytes. Human ES (“hES”) cells differentiated into albumin-expressing cells by the methods of the invention had albumin mRNA levels approximately as high as 1% of adult human hepatocytes at day 43 of culture, indicating that liver protein expression could be sustained for over a month in culture. Expression of other hepatocyte-specific proteins is also induced, at the levels reported in detail in the Examples.

Similarly, the culture conditions defined herein permit maintaining differentiated hepatocytes in culture for extended periods. For example, mouse hepatocytes extracted from mouse livers and cultured in the optimal culture conditions for 35 days showed levels of albumin mRNA that were 22% those of newly isolated cells, and continued to show appropriate hepatocyte phenotype both by light microscopy and by more detailed electron microscopy. Since rodent hepatocytes dedifferentiate quickly when placed in normal culture conditions, the present results show that the optimal culture conditions defined herein permit long term expression of a normal hepatocyte phenotype. Preferably, the long term expression (or expression “for an extended period”) is at least 20 days, more preferably 30 days, even more preferably, 35 days, 40 days, 43 days, 50 days, 54 days, or more.

We have further found that the expression of hepatocyte-specific proteins discussed above can be further improved by adding sodium butyrate and dimethyl sulfoxide (“DMSO”) to the medium. These compounds were added to medium and the gene expression of three separate hepatocyte related compounds—human albumin, α1-antitrypsin, and transferrin—was determined. As reported in the Examples, in each case, the gene expression was markedly increased. Sodium butyrate is known to be an erthyroid differentiation inducer. See, e.g., Yang et al., J Biol Chem, 276(28):25742-25752 (2001). DMSO and n-butyrate are known to promote differentiation characteristics in certain cells, including hepatocytes. Gladhaug et al., Anticancer Res, 9(6):1587-92 (1989). Usually, these compounds have been used to explore their effect in reducing proliferation of cancer cells.

Preferably, the amount of sodium butyrate added is from 0.25-10 mM, with 1-5 mM being preferred, 2-4 mM more preferred and about 2.5 mM being still more preferred. The DMSO can be added from about 0.1% to about 10%, with 0.5-4% being preferred, 0.5-2.5% being more preferred and about 1% being most preferred. The sodium butyrate and DMSO can be added independently, but preferably are added together.

Thus, in some embodiments, the methods of the invention permit increasing the expression of hepatocyte-related compounds by introducing sodium butyrate and DMSO in addition to human insulin and dexamethasone in the culture medium. Growth and differentiation factors other than human insulin, dexamethasone, sodium butyrate, and DMSO are unnecessary and are preferably omitted.

B. Transducing Differentiated Cells with Liver-Specific Lentivirus

Typically, not all the ES cells in a population treated by the methods of the invention will differentiate into hepatocytes or hepatocyte-like cells. The cells which have differentiated along the desired path can be identified by transducing them with lentivirus vectors containing genes encoding marker proteins. The genes are engineered to have their expression driven by hepatocyte-specific promoters. The cells expressing the proteins will be hepatocytes or hepatocyte-like cells, and can be identified compared to other cells in the population by the presence of the marker protein. A number of marker proteins are known in the art and are suitable for use in the methods of the invention. Preferred markers include green and red fluorescent protein. Antibiotic resistance genes can also be used as markers. The population of cells are transduced and then exposed to the antibiotic at levels that will kill cells not expressing the resistance gene, but that will not kill cells expressing the antibiotic resistance gene. The living cells are therefore selected for being hepatocytes or hepatocyte-like cells.

The transduction is preferably performed with a lentivirus vector. The vector is preferably engineered to be inactivated. In a preferred form, the inactivation is by deleting essential promoter/enhancer sequences of the long terminal repeat (LTR), resulting in transcriptional inactivation of the integrated virus. Lentiviruses inactivated by this technique are known in the art as “self-inactivating vectors.” See, e.g., Zufferey R., et al., “Self-inactivating lentivirus vector for safe and efficient in vivo gene delivery,” J Virol. 72(12):9873-80 (1998); Miyoshi H, et al., “Development of a self-inactivating lentivirus vector,” J Virol. 72(10):8150-7 (1998).

It is further preferred that the lentiviral vector include the post-transcriptional regulatory element of the woodchuck hepatitis virus (WPRE) and a central polypurine tract (CPPT), because these elements have been shown to enhance lentiviral gene expression in several cell lines, including stem cells. The WPRE is described by, for example, Donello et al., J Virol. 72:5085-92 (1998) and Zufferey, et al., J. Virol. 73(4):2886-92 (1999). As is known in the art, lentiviruses contain a central copy of the polypurine tract (CPPT) at which synthesis of the downstream plus strand is initiated. Inclusion of the upstream CPPT element enhances nuclear import of the derived vector genome and improves transduction efficiency. See generally, Robson and Telesnitsky, J. Virol., 74(22):10293-10303 (2000). Lentiviral vectors containing the HIV-1 CPPT and the WPRE have been constructed and have been shown to transduce non-dividing hepatocytes in vivo. See, VandenDriessche et al., Blood, 100(3):813-22 (2002). The ability to transduce non-dividing hepatocytes obviated the need to induce liver cell proliferation before transduction could occur. Id.

To achieve liver-specific transgene expression, a promoter for a protein that is highly expressed in the liver preferentially to other tissues, such as the human α1-antitrypsin promoter, is selected for use as a regulatory element for the transgene whose expression is desired. The human gene encoding α1-antitrypsin (“1AT”) is highly expressed in the liver and in cultured hepatoma cells and only to a lesser extent in macrophages, where transcription originates from a separate upstream promoter. See, e.g., Rollini and Fournier, Nucl Acids Res, 28(8):1767-1777 (2000). Thus, the promoter driving 1AT expression in liver cells results in high expression level in hepatocytes and in ES cells differentiated into hepatocyte protein-expressing cells by the methods of the invention. Promoters for other proteins expressed only, or preferentially, in hepatocytes, such as albumin, could, of course, be used in place of the 1AT promoter to drive the expression of the protein.

An exemplar use of a self inactivating lentiviral vector to transduce differentiated ES cells to express a marker protein is described in the Examples. It is expected that the same technique can be used to introduce and to express other marker proteins in such cells. In a preferred use, the transduced cells are then sorted by techniques known in the art, such as fluorescence activated cell sorting, to provide a population of hepatocytes or hepatocyte-like cells, or in which a high percentage of the cells are hepatocytes or hepatocyte-like cells.

C. Uses

Cells differentiated from ES cells, such as human ES cells, by the methods and compositions of the invention, and cells expressing proteins expressed exclusively or preferentially by hepatocytes, maintained or enhanced by the methods and compositions of the invention, have multiple uses. A number of uses are discussed in detail in the sections below; nevertheless, several will be briefly mentioned now.

The liver is a critical organ, and toxicity to the liver is a critical failing for a drug candidate. The ability to induce differentiation of ES cells into cells of the hepatic lineage, and the ability to maintain hepatocytes as differentiated cells for extended periods, permits the use of differentiated hepatocytes for in vitro screening of drug candidates for hepatotoxicity. This increased ability to perform in vitro screening can reduce the amount of pre-clinical testing in animal models, and reduce the consequent difficulties with animal rights advocates. Additionally, such screening may improve the chance that agents which have hepatotoxicity over time can be caught before they proceed through into clinical trials. This may help avoid some of the high expense and risk of harm to patients associated with agents that have hepatotoxicity which is not detected until late stage clinical trials or with an approved drug. Thus, cells differentiated from ES cells, such as human ES cells, by the methods and compositions of the invention, and hepatocytes maintained by the methods and compositions of the invention have important in vitro applications. Other in vitro applications are discussed in detail below.

In a further group of important embodiments, cells differentiated from ES cells, such as human ES cells, by the methods and compositions of the invention, and differentiated cells and cells expressing proteins expressed exclusively or preferentially by hepatocytes, maintained by the methods and compositions of the invention, can be used in liver assist devices, such as extracorporeal liver assist devices, in cases in which a patient's liver has lost much of its function. This is especially useful in cases of acute liver failure or fulminant liver disease, in which the liver loses function abruptly, giving the patient a period of only days, weeks, or months to find a suitable transplant. In such cases, the hepatocytes can be cultured in the liver assist device to help detoxify the patient's blood and to supply at least some of necessary liver proteins as a bridge until a suitable liver becomes available for transplantation.

Further, the cells differentiated from ES cells, such as human ES cells, by the methods and compositions of the invention, expressing proteins expressed exclusively or preferentially by hepatocytes can be used as an in vitro source of these proteins for use as reagents or for clinical use.

### DEFINITIONS

Units, prefixes, and symbols are denoted in their Systéme International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety. Terms not defined herein have their ordinary meaning as understood by a person of skill in the art.

For convenience, the following abbreviations are used at various points herein. “aFGF”=acidic fibroblast growth factor; “α1-AT”=alphal-antitrypsin; “bFGF”=basic fibroblast growth factor; “BI”=bovine insulin; “CK19”=cytokeratin 19; “DXM”=dexamethasone; “DMEM”=Dulbecco's modified Eagle's medium; “EBs”=embryoid bodies; “ES cells”=embryonic stem cells; “FBS”=fetal bovine serum; “GAPDH”=glyceraldehyde phosphate dehydrogenase; “GGT”=γ-glutamyl transferase; “G6P”=glucose-6-phosphatase; “hEGF”=human epidermal growth factor; “HGF”=hepatocyte growth factor; “HI”=human insulin; “IMDM”=Iscove's modified Dulbecco's medium; “LIF”=leukemia inhibitory factor; “MAPC”=multipotent adult progenitor cells; “mEGF”=mouse epidermal growth factor; “NGF”=nerve growth factor; “OnM”=oncostatin M; “RA”=all-trans-retinoic acid; “WME”=Williams' medium E.

“Growth factors,” along with cytokines and “hormones, are “secreted soluble factors that elicit their biological effects at picomolar concentrations by binding to receptors on target cells. Growth factors tend to be produced constitutively.” Goldsby et al., Kuby Immunology, 4th Ed., W. H. Freeman and Co., New York (2000), at page 304. The term “growth factor” tends to be used with respect to factors that induce or promote cell proliferation.

As used herein, a “differentiation factor” is a factor that induces or promotes differentiation from a precursor cell to a more differentiated cell type, including differentiation of a precursor cell into a terminally differentiated cell. Many cytokines are considered to have both proliferation inducing and differentiation inducing activity; thus, there is not a rigid division between growth factors and differentiation factors.

The phrase “consisting essentially of” is used, with respect to a growth factor, in its conventional meaning in U.S. patent law. Specifically, with respect to media “consisting essentially of” (a) the growth factors insulin and dexamethasone or, (b) insulin, dexamethasone, sodium butyrate, and DMSO, other growth factors are not present or, if other growth factors are present, they are present in amounts that do not reduce the ability of the differentiated ES cells to produce hepatocyte-specific proteins by more than 50% compared to like ES cells differentiated in the presence of the growth factors listed in (a) or (b), above, but not the other growth factors, more preferably by not more than 40%, even more preferably by not more than 30%, still more preferably by not more than 20% and most preferably by not more than 10% compared to like ES cells differentiated in the presence of the growth factors listed in (a) or of (b), above, but not the other growth factors. For purposes of this application, dexamethasone, DMSO and sodium butyrate are considered to be growth factors based on their effects on differentiation of ES cells and on maintenance of primary hepatocytes, as reported herein.

“Along a hepatocyte lineage” has its usual meaning in the art in directing an embryonic stem cell to differentiate along a pathway leading to production of proteins considered specific to hepatocytes.

A “primary hepatocyte” is a hepatocyte isolated directly from an animal, such as a human.

The term “hepatocyte like cells” as used herein includes reference to cells differentiated from ES cells, such as human ES cells, by the methods and compositions of the invention, which express proteins that are expressed exclusively or preferentially by primary hepatocytes, unless otherwise required by context. For clarity, it is noted that the term does not encompass cells of other differentiated cell types (e.g., adipocytes) which have been transduced to recombinantly express a protein normally expressed only by hepatocytes.

The terms “hepatocyte lineage” cell, “hepatoblastoid” cell and “hepatoembryoid” cell may be used in reference to the differentiated cells of this invention, obtained by differentiating pluripotent cells in the manner described. The differentiated cells have at least one of a variety of distinguishing phenotypic characteristics of known hepatocyte precursor cells, hepatoblasts, and hepatocytes, as provided later in this disclosure. By the use of these terms, no particular limitation is implied with respect to cell phenotype, cellular markers, cell function, or proliferative capacity, except where explicitly required.

A “hepatocyte precursor cell” or a “hepatocyte stem cell” is a cell that can proliferate and further differentiate into a hepatocyte, under suitable environmental conditions. Such cells may on occasion have the capacity to produce other types of progeny, such as oval cells, bile duct epithelial cells, or additional hepatocyte precursor cells.

“Proteins expressed exclusively or preferentially by hepatocytes” and “hepatocyte-specific protein” encompasses proteins that are produced exclusively by hepatocytes and proteins that are expressed by hepatocytes and by other tissues or organs, but whose expression in hepatocytes (as measured, for example, by mRNA transcripts) is at least 10 times higher lower than the expression in cells of other tissues, more preferably 20 times higher, and even more preferably in successive order, 30, 40, or 50 times higher. For example, transferrin is made by tissues other than the liver, but by mRNA transcript quantity, liver cells express almost 60 times more transferrin than cells of the next largest transferrin-expressing tissue type. See, e.g., Adrian et al., J Biol Chem., 265(22): 13344-13350 (1990); Idszerda et al., Proc Natl Acad Sci USA 83:3723-27 (1986). Other hepatocyte specific proteins include prothombin, liver enzymes such as alanine aminotransferase (“ALT”or “SGPT”), gamma-glutamyltranspeptidase, and fibrinogen. The amino acid sequences of these proteins and nucleic acid sequences encoding them are known, as are methods of recombinant expression (see, e.g., U.S. Pat. No. 6,037,457). In some preferred uses, the term refers to albumin, pre-albumin, glucose-6-phosphatase, and α1-antitrypsin. It is presumed that the person of skill is familiar with the proteins expressed by hepatocytes and the expression of those proteins relative to cells of other organs.

As used herein, “embryonic stem cells,” “ES cells” and “ESC” refer to pluripotent cells derived from pre-embryonic, embryonic, or fetal tissue at any time after fertilization, and have the characteristic of being capable under the right conditions of producing progeny of several different cell types. As defined for the purposes of this disclosure, ES cells are capable of producing progeny that are derivatives of all of the three germinal layers: endoderm, mesoderm, and ectoderm, according to a standard art-accepted test, such as the ability to form a teratoma in a suitable host. Human embryonic stem (hES) cells are described by Thomson et al., “Embryonic Stem Cell Lines Derived from Human Blastocysts”, Science 282:1145-1147 (1998). Embryonic stern cells from other primates, such as Rhesus monkeys, have also been described. See, e.g., Thomson et al., Proc. Natl. Acad. Sci. USA 92:7844 (1995). ES cells of non-primates, such as mice, have also been described.

ES cell cultures are said to be “essentially undifferentiated” when they display the morphology that clearly distinguishes them from differentiated cells of embryo or adult origin. ES cells typically have high nuclear/cytoplasmic ratios, prominent nucleoli, and compact colony formation with poorly discemable cell junctions, and are easily recognized by those skilled in the art. Colonies of undifferentiated cells can be surrounded by neighboring cells that are differentiated. Nevertheless, the essentially undifferentiated colony will persist when cultured under appropriate conditions, and undifferentiated cells constitute a prominent proportion of cells proliferating upon passaging of the cultured cells. Cell populations that contain any proportion of undifferentiated ES with these criteria can be used in this invention. Cell cultures described as essentially undifferentiated will typically contain at least about 20%, 40%, 60%, or 80% undifferentiated ES, in order of increasing preference.

A “growth environment” is an environment in which cells of interest will proliferate in vitro. Features of the environment include the medium in which the cells are cultured, the temperature, the partial pressure of O₂ and CO₂, and a supporting structure (such as a substrate on a solid surface) if present.

A “nutrient medium” is a medium for culturing cells containing nutrients that promote proliferation. The nutrient medium may contain any of the following in an appropriate combination: isotonic saline, buffer, amino acids, antibiotics, serum or serum replacement, and exogenously added factors. Numerous nutrient media are known in the art and many are commercially available.

A “conditioned medium” is prepared by culturing a first population of cells in a medium, and then harvesting the medium. The conditioned medium (along with anything secreted into the medium by the cells) may then be used to support the growth of a second population of cells.

“Restricted developmental lineage cells” are cells derived from embryonic tissue, typically by differentiation of ES cells. These cells are capable of proliferating and may be able to differentiate into several different cell types, but the range of phenotypes of their progeny is limited. Examples include: hematopoetic cells, which are pluripotent for blood cell types; neural precursors, which can generate glial cell precursors that progress to oligodendrocytes; neuronal restrictive cells, which progress to various types of neurons; and hepatocyte progenitors, which are pluripotent for hepatocytes and sometimes other liver cells, such as bile duct epithelium.

### CHARACTERISTICS OF DIFFERENTIATED CELLS

Cells can be characterized according to a number of phenotypic criteria. The criteria include but are not limited to the detection or quantitation of expressed cell markers, and enzymatic activity, and the characterization of morphological features and intercellular signaling.

Certain differentiated ES cells embodied in this invention have morphological features characteristic of hepatocytes. The features are readily appreciated by those skilled in evaluating such things, and include any or all of the following: a polygonal cell shape, a binucleate phenotype, the presence of rough endoplasmic reticulum for synthesis of secreted proteins, the presence of Golgi-endoplasmic reticulum lysosome complex for intracellular protein sorting, the presence of peroxisomes and glycogen granules, relatively abundant mitochondria, and the ability to form tight intercellular junctions resulting in creation of bile canalicular spaces. A number of these features present in a single cell is consistent with the cell being a member of the hepatocyte lineage. Unbiased determination of whether cells have morphologic features characteristic of hepatocytes can be made by coding micrographs of differentiated ES cells, adult or fetal hepatocytes, and one or more negative control cells, such as a fibroblast, or RPE (Retinal pigment epithelial) cells—then evaluating the micrographs in a blinded fashion, and breaking the code to determine if the differentiated ES cells are accurately identified.

Cells of this invention can also be characterized according to whether they express phenotypic markers characteristic of cells of the hepatocyte lineage. Cell markers useful in distinguishing liver progenitors, hepatocytes, and biliary epithelium are known in the art and can be found in such references as, for example, Sell & Zoran, Liver Stem Cells, R. G. Landes Co., Tex., 1997 and Grisham et al., p242 of “Stem Cells”, Academic Press, 1997).

It has been reported that hepatocyte differentiation requires the transcription factor HNF4α (Li et al., Genes Dev. 14:464, 2000). Markers independent of HNF-4α expression include α1-antitrypsin, α-fetoprotein, apoE, glucokinase, insulin growth factors 1 and 2, IGF-1 1 receptor, insulin receptor, and leptin. Markers dependent on HNF-4α expression include albumin, apoAI, apoAII, apoB, apoCIII, apoCII, aldolase B, phenylalanine hydroxylase, L-type fatty acid binding protein, transferrin, retinol binding protein, and erythropoietin (EPO).

Assessment of the level of expression of such markers can be determined in comparison with other cells. Positive controls for the markers of mature hepatocytes include adult hepatocytes of the species of interest, and established hepatocyte cell lines, such as the HepG2 line derived from a hepatoblastoma reported in U.S. Pat. No. 5,290,684. The reader is cautioned that permanent cell lines such as HepG2 may be metabolically altered, and fail to express certain characteristics of primary hepatocytes such as cytochrome p450. Cultures of primary hepatocytes may also show decreased expression of some markers after prolonged culture. Negative controls include cells of a separate lineage, such as an adult fibroblast cell line, or retinal pigment epithelial (RPE) cells.

Tissue-specific protein and oligosaccharide determinants can be detected using any suitable immunological technique—such as flow immunocytochemistry for cell-surface markers, immunohistochemistry (for example, of fixed cells or tissue sections) for intracellular or cell-surface markers, Western blot analysis of cellular extracts, and enzyme-linked immunoassay, for cellular extracts or products secreted into the medium. Expression of an antigen by a cell is said to be “antibody-detectable” if a significantly detectable amount of antibody will bind to the antigen in a standard immunocytochemistry or flow cytometry assay, optionally after fixation of the cells, and optionally using a labeled secondary antibody or other conjugate (such as a biotin-avidin conjugate) to amplify labeling.

The expression of tissue-specific markers can also be detected at the mRNA level by Northern blot analysis, dot-blot hybridization analysis, or by reverse transcriptase initiated polymerase chain reaction (RT-PCR) using sequence-specific primers in standard amplification methods. See U.S. Pat. No. 5,843,780 for further details. Sequence data for the particular markers listed in this disclosure can be obtained from public databases such as GenBank. Expression at the mRNA level is said to be “detectable” according to one of the assays described in this disclosure if the performance of the assay on cell samples according to standard procedures in a typical controlled experiment results in clearly discernable hybridization or amplification product. Expression of tissue-specific markers as detected at the protein or mRNA level is considered positive if the level is at least 2-fold, and preferably more than 10- or 50-fold above that of a control cell, such as an undifferentiated ES cell, a fibroblast, or other unrelated cell type.

Cells can also be characterized according to whether they display enzymatic activity that is characteristic of cells of the hepatocyte lineage. For example, assays for glucose-6-phosphatase activity are described by Bublitz (Mol Cell Biochem. 108:141, 1991); Yasmineh et al. (Clin. Biochem. 25:109, 1992); and Ockerman (Clin. Chim. Acta 17:201, 1968). Assays for alkaline phosphatase (ALP) and 5-nucleotidase (5′-Nase) in liver cells are described by Shiojiri (J. Embryol. Exp. Morph.62: 139, 1981). A number of laboratories that serve the research. and health care sectors provide assays for liver enzymes as a commercial service.

Cytochrome p450 is a key catalytic component of the mono-oxygenase system. It constitutes a family of hemoproteins responsible for the oxidative metabolism of xenobiotics (administered drugs), and many endogenous compounds. Different cytochromes present characteristic and overlapping substrate specificity. Most of the biotransforming ability is attributable by the cytochromes designated 1A2, 2A6, 2B6, 3A4, 2C9-11, 2D6, and 2E1 (Gomes-Lechon et al., pp 129-153 in “In vitro Methods in Pharmaceutical Research,” Academic Press, 1997).

A number of assays are known in the art for measuring cytochrome p450 enzyme activity. For example, cells can be contacted with a non-fluorescent substrate that is convertible to a fluorescent product by p450 activity, and then analyzed by fluorescence-activated cell counting (U.S. Pat. No. 5,869,243). Specifically, the cells are washed, and then incubated with a solution of 10 μM/L 5,6-methoxycarbonylfluorescein (Molecular Probes, Eugene Oreg.) for 15 min at 37° C. in the dark. The cells are then washed, trypsinized from the culture plate, and analyzed for fluorescence emission at about 520-560 nm. A cell is said to have the enzyme activity assayed for if the level of activity in a test cell is more than 2-fold, and preferably more than 10- or 100-fold above that of a control cell, such as a fibroblast.

The expression of cytochrome p450 can also be measured at the protein level, for example, using specific antibody in Western blots, or at the mRNA level, using specific probes and primers in Northern blots or RT-PCR. See Borlakoglu et al., Int. J. Biochem. 25:1659, 1993. Particular activities of the p450 system can also be measured: 7-ethoxycoumarin O-de-ethylase activity, aloxyresorufin O-de-alkylase activity, coumarin 7-hydroxylase activity, p-nitrophenol hydroxylase activity, testosterone hydroxylation, UDP-glucuronyltransferase activity, glutathione S-transferase activity, and others (reviewed in Gomes-Lechon et al., pp 411-431 in “In vitro Methods in Pharmaceutical Research,” Academic Press, 1997). The activity level can then be compared with the level in primary hepatocytes.

Assays are also available for enzymes involved in the conjugation, metabolism, or detoxification of small molecule drugs. For example, cells can be characterized by an ability to conjugate bilirubin, bile acids, and small molecule drugs, for excretion through the urinary or biliary tract. Cells are contacted with a suitable substrate, incubated for a suitable period, and then the medium is analyzed (by GCMS or other suitable technique) to determine whether conjugation product has been formed. Drug metabolizing enzyme activities include de-ethylation, dealkylation, hydroxylation, demethylation, oxidation, glucuroconjugation, sulfoconjugation, glutathione conjugation, and N-acetyl transferase activity (A. Guillouzo, pp 411-431 in “In vitro Methods in Pharmaceutical Research,” Academic Press, 1997). Assays include peenacetin de-ethylation, procainamide N-acetylation, paracetamol sulfoconjugation, and paracetamol glucuronidation (Chesne et al., pp 343-350 in “Liver Cells and Drugs”, A. Guillouzo ed. John Ubbey Eurotext, London, 1988).

Cells of the hepatocyte lineage can also be evaluated on their ability to store glycogen. A suitable assay uses Periodic Acid Schiff (PAS) stain, which does not react with mono- and disaccharides, but stains long-chain polymers such as glycogen and dextran. PAS reaction provides quantitative estimations of complex carbohydrates as well as soluble and membrane-bound carbohydrate compounds. Kirkeby et al. (Biochem. Biophys. Meth. 24:225, 1992) describe a quantitative PAS assay of carbohydrate compounds and detergents. van der Laarse et al. (Biotech Histochem. 67:303, 1992) describe a microdensitometric histochemical assay for glycogen using the PAS reaction. Evidence of glycogen storage is determined if the cells are PAS-positive at a level that is at least 2-fold, and preferably more than 10-fold above that of a control cell, such as a fibroblast. The cells can also be characterized by karyotyping according to standard methods.

ES cells differentiated according to this invention can have a number of the aforementioned features, including antibody-detectable expression of α1-antitrypsin (AAT) or albumin; absence of antibody-detectable expression of α-fetoprotein; RT-PCR detectable expression of asialoglycoprotein receptor (either the ASGR-1 or ASGR-2 isotype); evidence of glycogen storage; evidence of cytochrome p450 or glucose-6-phosphatase activity; and morphological features characteristic of hepatocytes. The more of these features that are present in a particular cell, the more it can be characterized as a cell of the hepatocyte lineage. Cells having at least 2, 3, 5, 7, or 9 of these features are increasingly more preferred. In reference to a particular cell population as may be present in a culture vessel or a preparation for administration, uniformity between cells in the expression of these features is often advantageous.

Other desirable features of differentiated cells of this invention are an ability to act as target cells in drug screening assays, and an ability to reconstitute liver function, both in vivo, and as part of an extracorporeal device.

### USES OF CELLS DIFFERENTIATED OR MAINTAINED BY THE METHODS OF THE INVENTION

This invention provides a method by which large numbers of cells of the hepatocyte lineage can be produced or can be maintained. These cell populations can be used for a number of important research, development, and commercial purposes.

A. Preparation of Expression Libraries and Specific Antibody

Cells maintained by this invention, such as primary hepatocytes, can be used to prepare a cDNA library relatively uncontaminated with cDNA preferentially expressed in cells from other lineages. For example, the cells are collected by centrifugation at 1000 rpm for 5 min, and then mRNA is prepared from the pellet by standard techniques (e.g., Sambrook et al., Molecular Cloning: A Laboratory Manual, CSHL Press, Woodbury, N.Y. (3rd. Ed., 2001)). After reverse transcribing into cDNA, the preparation can be subtracted with cDNA from any or all of the following cell types: sinusoidal endothelial cells, bile duct epithelium, or other cells of undesired specificity, thereby producing a select cDNA library, reflecting expression patterns that are representative of mature hepatocytes, hepatocyte precursors or both.

The differentiated cells of this invention can also be used to prepare antibodies that are specific for hepatocyte markers, progenitor cell markers, markers that are specific for hepatocyte precursors, and other antigens that may be expressed on the cells. The cells of this invention provide an improved way of raising such antibodies because they are relatively enriched for particular cell types compared with ES cell cultures. Polyclonal antibodies can be prepared by injecting a vertebrate with cells of this invention in an immunogenic form. Production of monoclonal antibodies is described in such standard references as Harlow and Lane, Using Antibodies: A Laboratory Manual, Cold Spring Harbor Publications, New York (1999), U.S. Pat. Nos. 4,491,632, 4,472,500 and 4,444,887, and Methods in Enzymology 73B:3 (1981). Other methods of obtaining specific antibody molecules (optimally in the form of single-chain variable regions) involve contacting a library of immunocompetent cells or viral particles with the target antigen, and growing out positively selected clones. See Marks et al., New Eng. J. Med. 335:730, 1996, International Patent Applications WO 94/13804, WO 92/01047, WO 90/02809, and McGuiness et al., Nature Biotechnol. 14:1449, 1996. By positively selecting using ES cells of this invention, and negatively selecting using cells bearing more broadly distributed antigens (such as undifferentiated embryonic cells) or adult-derived stem cells, the desired specificity can be obtained. The antibodies in turn can be used to identify or rescue hepatocyte precursor cells of a desired phenotype from a mixed cell population, for purposes such as costaining during immunodiagnosis using tissue samples, and isolating such cells from mature hepatocytes or cells of other lineages.

B. Genomics

Differentiated ES cells are of interest to identify expression patterns of transcripts and newly synthesized proteins that are characteristic for hepatocyte precursor cells, and may assist in directing the differentiation pathway or facilitating interaction between cells. Expression patterns of the differentiated cells are obtained and compared with control cell lines, such as undifferentiated ES cells, other types of committed precursor cells (such as ES cells differentiated towards other lineages, hematopoietic stem cells, precursor cells for other mesoderm-derived tissue, precursor cells for endothelium or bile duct epithelium, hepatocyte stem cells obtained from adult tissues, or ES cells differentiated towards the hepatocyte lineage using alternative reagents or techniques).

Suitable methods for comparing expression at the protein level include the immunoassay or immunohistochemistry techniques describe earlier. Suitable methods for comparing expression at the level of transcription include methods of differential display of mRNA (Liang, Peng, et al., Cancer Res. 52:6966, 1992), and matrix array expression systems (Schena et al., Science 270:467, 1995; Eisen et al., Methods Enzymol. 303:179, 1999; Brown et al., Nat. Genet. 21 Suppl 1:33, 1999).

The use of microarray in analyzing gene expression is reviewed by, e.g., Fritz et al Science 288:316, 2000; “Microarray Biochip Technology”, M. Schena ed., Eaton Publishing Company; “Microarray analysis”, Gwynne & Page, Science (Aug. 6, 1999 supplement); Pollack et al., Nat Genet 23:41, 1999; and Gerhold et al., Trends Biochem. Sci. 24:168, 1999. Systems and reagents for performing microarray analysis are available commercially from companies such as Affymetrix, Inc., Santa Clara, Calif.; Gene Logic Inc., Columbia, Md.; Hyseq Inc., Sunnyvale, Calif.; Molecular Dynamics Inc., Sunnyvale, Calif.; and Nanogen, San Diego, Calif.

Solid-phase arrays are manufactured by attaching the probe at specific sites either by synthesizing the probe at the desired position, or by presynthesizing the probe fragment and then attaching it to the solid support. A variety of solid supports can be used, including glasses, plastics, ceramics, metals, gels, membranes, paper, and beads of various composition. U.S. Pat. No. 5,445,934 discloses a method of on-chip synthesis, in which a glass slide is derivatized with a chemical species containing a photo-cleavable protecting group. Each site is sequentially deprotected by irradiation through a mask, and then reacted with a DNA monomer containing a photoprotective group. Methods for attaching a presynthesized probe onto a solid support include adsorption, ultra violet linking, and covalent attachment. In one example, the solid support is modified to carry an active group, such as hydroxyl, carboxyl, amine, aldehyde, hydrazine, epoxide, bromoacetyl, maleimide, or thiol groups through which the probe is attached (U.S. Pat. Nos. 5,474,895 and 5,514,785).

The probing assay is typically conducted by contacting the array by a fluid potentially containing the nucleotide sequences of interest under suitable conditions for hybridization, and then determining any hybrid formed. For example, mRNA or DNA in the sample is amplified in the presence of nucleotides attached to a suitable label, such as the fluorescent labels Cy3 or Cy5. Conditions are adjusted so that hybridization occurs with precise complementary matches or with various degrees of homology, as appropriate. The array is then washed, and bound nucleic acid is determined by measuring the presence or amount of label associated with the solid phase. Different samples can be compared between arrays for relative levels of expression, optionally standardized using genies expressed in most cells of interest, such as a ribosomal or house-keeping gene, or as a proportion of total polynucleotide in the sample. Alternatively, samples from two or more different sources can be tested simultaneously on the same array, by preparing the amplified polynucleotide from each source with a different label.

An exemplary method is conducted using a Genetic Microsystems array generator, and an Axon Genepix™ Scanner. Microarrays are prepared by first amplifying cDNA fragments encoding marker sequences to be analyzed in a 96 or 384 well format. The cDNA is then spotted directly onto glass slides at a density as high as >5,000 per slide. To compare mRNA preparations from two cells of interest, one preparation is converted into Cy3-labeled cDNA, while the other is converted into Cy5-labeled cDNA. The two cDNA preparations are hybridized simultaneously to the microarray slide, and then washed to eliminate non-specific binding. Any given spot on the array will bind each of the cDNA products in proportion to abundance of the transcript in the two original mRNA preparations. The slide is then scanned at wavelengths appropriate for each of the labels, the resulting fluorescence is quantified, and the results are formatted to give an indication of the relative abundance of mRNA for each marker on the array.

Identifying expression products for use in characterizing and affecting differentiated cells of this invention involves analyzing the expression level of RNA, protein, or other gene product in a first cell type, such as a ES cell differentiated along the hepatocyte lineage, analyzing the expression level of the same product in a control cell type, comparing the relative expression level between the two cell types, (typically normalized by total protein or RNA in the sample, or in comparison with another gene product expected to be expressed at a similar level in both cell types, such as a house-keeping gene), and identifying products of interest based on the comparative expression level.

Products will typically be of interest if their relative expression level is at least about 2-fold, 10-fold, or 100-fold elevated (or suppressed) in differentiated ES cells of this invention, in comparison with the control. This analysis can optionally be computer-assisted, by marking the expression level in each cell type on an independent axis, wherein the position of the mark relative to each axis is in accordance with the expression level in the respective cell, and then selecting a product of interest based on the position of the mark. Alternatively, the difference in expression between the first cell and the control cell can be represented on a color spectrum (for example, where yellow represents equivalent expression levels, red indicates augmented expression and blue represents suppressed expression). The product of interest can then be selected based on the color representing expression of one marker of interest, or based on a pattern of colors representing a plurality of markers.

C. Drug Screening

Differentiated ES cells of this invention can be used to screen for factors (such as solvents, small molecule drugs, peptides, polynucleotides, and the like) or environmental conditions (such as culture conditions or manipulation) that affect the characteristics of differentiated cells of the hepatocyte lineage.

In some applications, ES cells (differentiated or undifferentiated) are used to screen factors that promote maturation of cells along the hepatocyte lineage, or promote proliferation and maintenance of such cells in long-term culture. For example, candidate hepatocyte maturation factors or growth factors are tested by adding them to ES cells in different wells, and then determining any phenotypic change that results, according to desirable criteria for further culture and use of the cells.

Particular screening applications of this invention relate to the testing of pharmaceutical compounds in drug research. Such applications are well known in the art, as exemplified in “In vitro Methods in Pharmaceutical Research”, Academic Press, 1997, and U.S. Pat. No. 5,030,015. In this invention, ES cells that have differentiated to the hepatocyte lineage play the role of test cells for standard drug screening and toxicity assays, as have been previously performed on hepatocyte cell lines or primary hepatocytes in short-term culture. Assessment of the activity of candidate pharmaceutical compounds generally involves combining the differentiated cells of this invention with the candidate compound, determining any change in the morphology, marker phenotype, or metabolic activity of the cells that is attributable to the compound (compared with untreated cells or cells treated with an inert compound), and then correlating the effect of the compound with the observed change. The screening may be done either because the compound is designed to have a pharmacological effect on liver cells, or because a compound designed to have effects elsewhere may have unintended hepatic side effects. Two or more drugs can be tested in combination (by combining with the cells either simultaneously or sequentially), to detect possible drug-drug interaction effects.

In some applications, compounds are screened initially for potential hepatotoxicity (Castell et al., pp 375-410 in “In vitro Methods in Pharmaceutical Research,” Academic Press, 1997). Cytotoxicity can be determined in the first instance by the effect on cell viability, survival, morphology, and leakage of enzymes into the culture medium. More detailed analysis is conducted to determine whether compounds affect cell function (such as gluconeogenesis, ureogenesis, and plasma protein synthesis) without causing toxicity. Lactate dehydrogenase (LDH) is a good marker because the hepatic isoenzyme (type V) is stable in culture conditions, allowing reproducible measurements in culture supernatants after 12-24 hour incubation. Leakage of enzymes such as mitochondrial glutamate oxaloacetate transaminase and glutamate pyruvate transaminase can also be used. Gomez-Lechon et al. (Anal. Biochem. 236:296, 1996) describe a microassay for measuring glycogen, which can be applied to measure the effect of pharmaceutical compounds on hepatocyte gluconeogenesis.

Other current methods to evaluate hepatotoxicity include determination of the synthesis and secretion of albumin, cholesterol, and lipoproteins; transport of conjugated bile acids and bilirubin; ureagenesis; cytochrome p450 levels and activities; glutathione levels; ATP, ADP, and AMP metabolism; intracellular K⁺ and Ca²⁺ concentrations; the release of nuclear matrix proteins or oligonucleosomes; and induction of apoptosis (indicated by cell rounding, condensation of chromatin, and nuclear fragmentation). DNA synthesis can be measured as [³H]-thymidine or BrdU incorporation. Effects of a drug on DNA synthesis or structure can be determined by measuring DNA synthesis or repair. [³H]-thymidine or BrdU incorporation, especially at unscheduled times in the cell cycle, or above the level required for cell replication, is consistent with a drug effect. Unwanted effects can also include unusual rates of sister chromatid exchange, determined by metaphase spread. Thereader is referred to A. Vickers (pp 375-410 in “In vitro Methods in Pharmaceutical Research,” Castell and Gmez-Lechn, eds., Academic Press, 1996) for further elaboration.

D. Production of Liver Proteins

ES cells differentiated by the methods of the invention or primary hepatocytes maintained in culture by the methods of the invention can be used to produce liver proteins for use as reagents or therapeutics. The proteins are secreted by the cells into the medium, and the proteins can then be isolated and purified by protocols known in the art.

Currently, liver proteins are typically prepared from human sera. For example, albumin for clinical use is commonly obtained from human venous plasma using the Cohn cold ethanol fractionation process. Since the protein is derived from human plasma, the albumin solution is heated for 10 hours at 60° C. to reduce the likelihood of the presence of viable hepatitis or other viruses.

The ability to obtain proteins, such as albumin, from cultivated cells uninfected by known diseases can therefore reduce the need to treat the protein without reducing patient safety.

E. Restoration or Support of Liver Function

This invention also provides for the use of differentiated ES cells to restore a degree of liver function to a subject needing such therapy, perhaps due to an acute, chronic, or inherited impairment of liver function.

To determine the suitability of differentiated ES cells for therapeutic applications, the cells can first be tested in a suitable animal model. At one level, cells are assessed for their ability to survive and maintain their phenotype in vivo. Differentiated ES cells are administered to immunodeficient animals (such as SCID mice, or animals rendered immunodeficient chemically or by irradiation) at a site amenable for further observation, such as under the kidney capsule, into the spleen, or into a liver lobule. Tissues are harvested after a period of a few days to several weeks or more, and assessed as to whether differentiated ES cells are still present. This can be performed by providing the administered cells with a detectable label (such as green fluorescent protein, or β-galactosidase); or by measuring a constitutive marker specific for the administered cells. Where differentiated ES cells are being tested in a rodent model, the presence and phenotype of the administered cells can be assessed by immunohistochemistry or ELISA using human-specific antibody, or by RT-PCR analysis using primers and hybridization conditions that cause amplification to be specific for human polynucleotide sequences. Suitable markers for assessing gene expression at the mRNA or protein level are known in the art. General descriptions for determining the fate of hepatocyte-like cells in animal models is provided in Grompe et al. (Sem. Liver Dis. 19:7, 1999); Peeters et al., (Hepatology 25:884, 1997;) and Ohashi et al. (Nature Med. 6:327, 2000).

At another level, differentiated ES cells are assessed for their ability to restore liver function in an animal lacking full liver function. Braun et al. (Nature Med. 6:320, (2000)) outline a model for toxin-induced liver disease in mice transgenic for the HSV tk gene. Rhim et al. (Proc. Natl. Acad. Sci. USA 92:4942, (1995)) and Lieber et al. (Proc. Natl. Acad. Sci. USA 92:6210, (1995)) outline models for liver disease by expression of urokinase. Mignon et al. (Nature Med. 4:1185, 1998) outline liver disease induced by antibody to the cell-surface marker Fas. Overturf et al. (Human Gene Ther. 9:295, 1998) have developed a model for Hereditary Tyrosinemia Type I in mice by targeted disruption of the Fah gene. The animals can be rescued from the deficiency by providing a supply of 2-(2-nitro-4-fluoro-methyl-benzyol)-1,3-cyclohexanedione (NTBC), but develop liver disease when NTBC is withdrawn. Acute liver disease can be modeled by 90% hepatectomy (Kobayashi et al., Science 287:1258, 2000). Acute liver disease can also be modeled by treating animals with a hepatotoxin such as galactosamine, CCl₄, or thioacetamide. Chronic liver diseases such as cirrhosis can be modeled by treating animals with a sub-lethal dose of a hepatotoxin long enough to induce fibrosis (Rudolph et al., Science 287:1253, 2000). Assessing the ability of differentiated cells to reconstitute liver function involves administering the cells to such animals, and then determining survival over a 1 to 8 week period or more, while monitoring the animals for progress of the condition. Effects on hepatic function can be determined by evaluating markers expressed in liver tissue, cytochrome p450 activity, and blood indicators, such as alkaline phosphatase activity, bilirubin conjugation, and prothrombin time), and survival of the host. Any improvement in survival, disease progression, or maintenance of hepatic function according to any of these criteria relates to effectiveness of the therapy, and can lead to further optimization.

This invention includes differentiated cells that can be encapsulated or used as a part of a bioartificial liver device (sometimes known as a “Liver Assist Device”). Various forms of encapsulation are described in “Cell Encapsulation Technology and Therapeutics”, Kuhtreiber et al. eds., Birkhauser, Boston Mass., 1999. Differentiated cells of this invention can be encapsulated according to such methods for use either in vitro or in vivo.

Bioartificial organs for clinical use are designed to support an individual with impaired liver function—either as a part of long-term therapy, or to bridge the time between a fulminant hepatic failure and hepatic reconstitution or liver transplant. Bioartificial liver devices are reviewed by Macdonald et al., pp. 252-286 of “Cell Encapsulation Technology and Therapeutics”, op cit., and exemplified in U.S. Pat. Nos. 5,290,684, 5,624,840, 5,837,234, 5,853,717, and 5,935,849. Suspension-type bioartificial livers comprise cells suspended in plate dialysers, or microencapsulated in a suitable substrate, or attached to microcarrier beads coated with extracellular matrix. Alternatively, hepatocytes can be placed on a solid support in a packed bed, in a multiplate flat bed, on a microchannel screen, or surrounding hollow fiber capillaries. The device has inlet and outlet through which the subject's blood is passed, and sometimes a separate set of ports for supplying nutrients to the cells.

Current proposals for such liver support devices involve hepatocytes from a xenogeneic source, such as a suspension of porcine hepatocytes, because of the paucity of available primary human hepatocytes. Xenogeneic tissue sources raise regulatory concerns regarding immunogenicity and possible cross-species viral transmission.

The present invention provides a system for generating preparative cultures of human cells. Differentiated pluripotent stem cells are prepared according to the methods described earlier, and then plated into the device on a suitable substrate, such as a matrix of Matrigel.RTM. or collagen. The efficacy of the device can be assessed by comparing the composition of blood in the afferent channel with that in the efferent channel—in terms of metabolites removed from the afferent flow, and newly synthesized proteins in the efferent flow.

Devices of this kind can be used to detoxify a fluid such as blood, wherein the fluid comes into contact with the differentiated cells of this invention under conditions that permit the cell to remove or modify a toxin in the fluid. The detoxification will involve removing or altering at least one ligand, metabolite, or other compound (either natural and synthetic) that is usually processed by the liver. Such compounds include but are not limited to bilirubin, bile acids, urea, heme, lipoprotein, carbohydrates, transferrin, hemopexin, asialoglycoproteins, hormones like insulin and glucagon, and a variety of small molecule drugs. The device can also be used to enrich the efferent fluid with synthesized proteins such as albumin, acute phase reactants, and unloaded carrier proteins. The device can be optimized so that a variety of these functions are performed, thereby restoring as many hepatic functions as are needed. In the context of therapeutic care, the device processes blood flowing from a patient in hepatocyte failure, and then the blood is returned to the patient.

The properties of cell differentiated or maintained by means of the present invention are particularly useful in liver assist devices (LAD). For the most part, the cells may be used in any device which provides a means for culturing the cells, as well as a means for separating the cells from blood which will be passed through the device. Membranes or capillaries are available in the literature for use which allow for the crossover of toxic solutes from the blood to the cells as well as the diffuision of vital metabolites provided by the cells across the membrane into the blood. The permiselective or semipermeable membrane additionally provides a mechanical barrier against the immune system. For the most part, a membrane or capillary will be used which features a molecular weight cutoff from about 20,000 daltons up to about 80,000 daltons, generally about 30,000 daltons to about 50,000 daltons. However, it may be preferable to utilize a membrane with pore sizes from about 0.1μ. to about 0.3μ, usually about 0.2μ. A pore size in this range will exclude cellular elements yet still allow proteins and protein complexes to pass through. Thus, the serum protein deficiencies of FHF can be ameliorated.

Generally, the cells are grown in the liver assist device. After growth of the cells, the subject's blood is passed through the device, and dissolved molecular species (e.g., bilirubin) diffuse through the membrane and are taken up and metabolized by the cells. The devices are typically employed in extracorporeal blood processing. Generally, the devices are designed to house the cells in a blood-perfused device attached to the blood stream. Typically, the device is attached to the blood stream between an artery and a vein.

Several designs of liver assist devices are known in the literature. For example, devices have been described by Viles et al., U.S. Pat. Nos. 4,675,002 and 4,853,324; Jauregin, GB 2,221,857A; Wolf et al., International J. of Artificial Organs 2:97-103 (1979); Wolf et al., International J. of Artificial Organs 1:45-51 (1978); and Ehrlich et al., In Vitro 14:443-450 (1978). Preferred devices include the hollow fiber cartridge and similar perfusion devices. Typically, the cells are encapsulated in biomaterials such as alginate-polylysine membranes, as taught by Cai et al., Artificial Organs 12:388-393; Sun et al., Trans. Am. Soc. Artif. Intern. Organs Vol. XXXII:39-41 (1986); O'Shea et al., Biochimica Biophysica Acta 804:133-136 (1984); Sun et al., J. Controlled Release 2:137-141 (1985); and U.S. Pat. No. 4,391,909.

Bioreactors, such as hollow fiber cartridges, may be utilized as liver assist devices. See, for example, Heifetz et al., BioTechniques 7:192-199 (1989); and Donofrio, D. M., Amer. Biotech. Lab. Sept. 1989, Publication #940.

The cells of the present invention, when grown in a hollow fiber cartridge or similar perfusion device with capacities for high numbers of cells, can function as a perfused liver, allowing accurate assessment of human liver metabolism and replacement of liver-specific biological activities. Therefore, a perfusion device containing a culture of the disclosed cells is capable of functioning as a liver assist device. In one embodiment of this invention the LAD is extracorporeal, referring to its connection to arterial and venous circulation outside the body. An extracorporeal LAD (or ELAD) is particularly useful for providing temporary liver support for subjects suffering from FHF.

Hollow fiber cartridges are two-chamber units which reproduce the three-dimensional characteristics of normal organs (Knazek, R. H., Feder. Proc. 33:1978-1981 (1974); Ku, K. et al., Biotechnol. Bioeng. 23:79-95 (1983)). Culture or growth medium is circulated through the capillary space and cells are grown in the extracapillary space (Tharakan, J. P. et al., Biotechnol. Bioeng. 28:1605-1611 (1986). Such hollow fiber culture systems have been disclosed as useful for culture of hybridoma cells lines for the production of monoclonal antibodies (Altshulter, G. L. et al., Biotechnol. Bioeng. 28:646-658 (1986); Heifetz, H. H. et al., supra; Donofrio, D. M., supra. Further, a number of other cell types, including the liver cell lines PLC/PRF 5 and Reuber hepatoma, (McAleer, W. J. et al. J. Virol. Meth. 7:263-271 (1983); Wolf, C. F. W. (1982)) and pancreatic islet cells (Araki, Y. et al., Diabetes 34:850-854 (1985)) have been cultured in this manner.

Once a device has been chosen for use as a liver assist device, it is provided with the appropriate medium and an inoculation of cells. The devices are then maintained in a 37° C. room with constant recirculation of medium and constant inflow of fresh medium. For use with a hollow fiber cartridge of 1400 cm², the cartridge is provided with 150 ml/min of recirculated medium with a constant inflow of about 0.5 ml/min. A 1400 cm² cartridge is generally inoculated with about 1×10⁹ cells.

The function of the cells in the device can now be tested for the capability of the device to function as a liver assist device. This includes measurements of essential liver biological functions as discussed above. It will usually not be necessary to add additional oxygen to the system. However, the oxygen tension in the cultures can be determined and additional oxygen added if necessary. To vary the oxygen tension in cultures of the selected cell lines to determine the optimum oxygen level, cells can be grown in a continuous perfusion apparatus. The apparatus will consist of a recirculation pump, medium bottles, and a lid that fits on a standard culture dish. The medium is continually recycled over the surface of the cells and back into the medium container where it can be gassed. Medium is gassed with preparations containing between 4% and 20% oxygen, 5% CO₂ and the remainder nitrogen. In this way, the cells can be maintained in the appropriate atmosphere such that the effect of the gas mixture can be determined. Growth rate may be determined by monitoring total cell protein per well.

ATP, ADP and AMP may be measured as described by Lundin et al., Meth. Enzymol. 133:27-41 (1986), using firefly luciferase. The ratio of NAD/NADH can be calculated from the ratio of lactate to pyruvate across tactic dehydrogenase and from the ratio of malate to oxaloacetate across malate dehydrogenase. The concentrations of these metabolites can be determined by methods set forth in Methods of Enzymatic Analysis, H. U. Bergmyer, ed., 3rd ed., Verlag Chemie, Weinheim, Vol. VI, pp. 570-588. The ratio of NADP/NADPH may be calculated from the ratio of isocitrate to alpha-ketoglutarate across isocitrate dehydrogenase and from the ratio of malate to pyruvate across malic enzyme. The determination of these metabolites is also set forth in Bergmyer, supra. Energy change may be calculated from the equation. (ATP+0.5 ADP)/(ATP+ADP+AMP).

Besides looking at the oxygen dependence of the liver assist device, the devices may also be characterized with respect to their ability to simulate an isolated, perfused human liver. This includes testing the device for glucose and urea synthesis, bilirubin uptake and conjugation, and clotting factor biosynthesis as described above. Urea may be quantitated using a coupled glutamate dehydrogenase/urease assay. Glucose may be determined using a dye-coupled glucose oxidase assay. Suitable assays for determining urea and glucose levels are found in Bergmyer, supra.

The cell lines may also find use as bioartificial livers or liver supports. In this manner, the cells are encapsulated or grown in hollow fiber capillary membranes for use as a bioartificial organ. The encapsulated cells and vehicle capsules are then injected intraperitoneally into a subject.

Differentiated ES cells of this invention that demonstrate desirable functional characteristics in animal models (such as those described above) may also be suitable for direct administration to human subjects with impaired liver function. For purposes of hemostasis, the cells can be administered at any site that has adequate access to the circulation, typically within the abdominal cavity. For some metabolic and detoxification functions, it is advantageous for the cells to have access to the biliary tract. Accordingly, the cells are administered near the liver (e.g., in the treatment of chronic liver disease) or the spleen (e.g., in the treatment of fulminant hepatic failure). In one method, the cells administered into the hepatic circulation either through the hepatic artery, or through the portal vein, by infusion through an in-dwelling catheter. A catheter in the portal vein can be manipulated so that the cells flow principally into the spleen, or the liver, or a combination of both. In another method, the cells are administered by placing a bolus in a cavity near the target organ, typically in an excipient or matrix that will keep the bolus in place. In another method, the cells are injected directly into a lobe of the liver or the spleen.

The differentiated cells of this invention can be used for therapy of any subject in need of having hepatic function restored or supplemented. Human conditions that may be appropriate for such therapy include fulminant hepatic failure due to any cause, viral hepatitis, drug-induced liver injury, cirrhosis, inherited hepatic insufficiency (such as Wilson's disease, Gilbert's syndrome, or α1-antitrypsin deficiency), hepatobiliary carcinoma, autoimmune liver disease (such as autoimmune chronic hepatitis or primary biliary cirrhosis), and any other condition that results in impaired hepatic function. For human therapy, the dose is generally between about 10⁹ and 10¹¹ cells, and typically between about 5×10⁹ and 5×10¹⁰ cells, making adjustments for the body weight of the subject, nature and severity of the affliction, and the replicative capacity of the administered cells. Decisions as the mode of treatment and the appropriate dose are made by the managing physician in light of the factors above, in the exercise of clinical judgment.

The following examples provided as further non-limiting illustrations of particular embodiments of the invention.

### EXAMPLES

### Example 1

### Materials and Methods

Culture of Mouse ES Cells

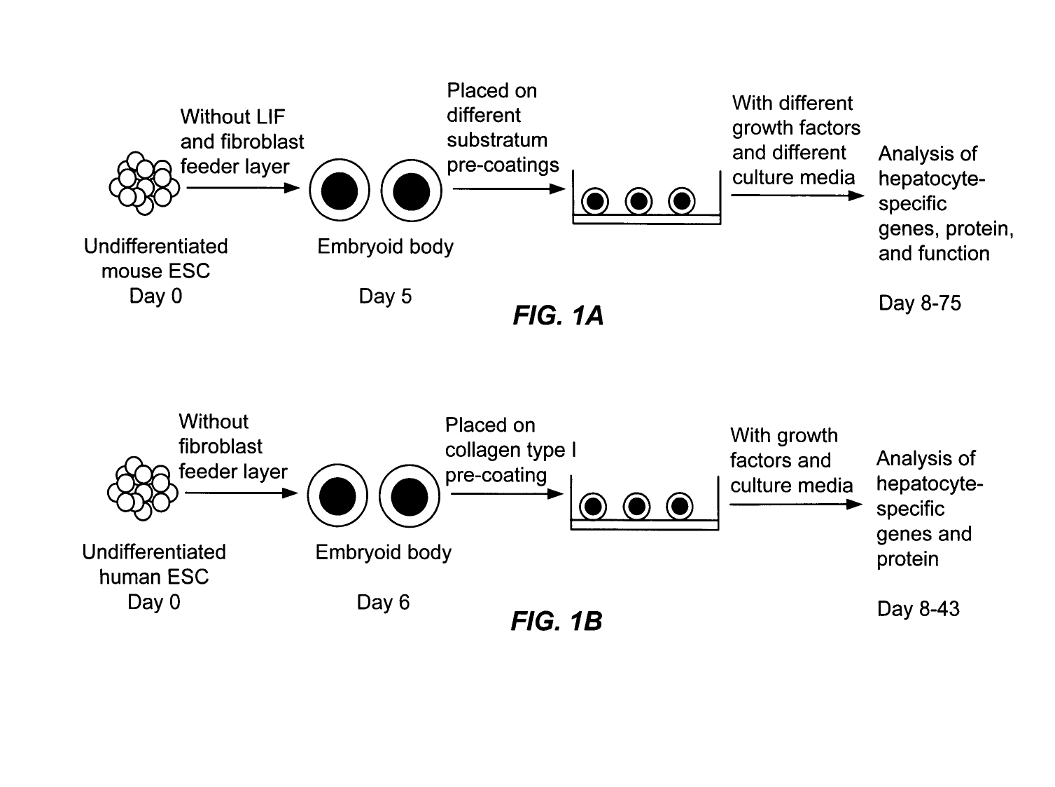

Mouse ES cell line ES-D3 and mouse STO fibroblasts were obtained from the American Type Culture Collection, Manassas, Va. ES cells were expanded on STO fibroblast feeder layers in Dulbecco's modified Eagle's medium (DMEM) (unless specified, cell culture supplies were from Invitrogen, Carlsbad, Calif.) containing 15% fetal bovine serum (FBS), 1 mM L-glutamine, 60 μM non-essential amino acid solution, 0.1 mM 2-mercaptoethanol, 10 mM HEPES (Sigma-Aldrich, Saint Louis, Mo.), 1400 U/ml leukemia inhibitory factor (LIF) (Chemicon International, Temecula, Calif.), and penicillin/streptomycin at standard concentrations. To prepare feeder layers, STO fibroblasts were cultured until confluent and treated with 10 μg/ml mitomycin C (Sigma-Aldrich) for 4 hours. Mitomycin C-treated STO fibroblasts were then re-seeded at a density of 7-8×10⁴/cm² one day before plating ES cells. As illustrated in FIG. 1A, differentiation of ES cells was initiated by seeding ES cells on non-coated 100-mm tissue culture dishes without an STO fibroblast feeder layer and LIF. These culture conditions stimulated the formation of non-attached embryoid bodies (EBs): Five days later, EBs were placed on different substratum pre-coated 6-well tissue culture plates in various media formulations, and the expression of hepatocyte-specific genes was examined at day 8-75 (FIG. 1A). Substrata used for pre-coating included gelatin, collagen type I (Sigma-Aldrich), collagen type IV, laminin, fibronectin and poly-D-lysine (Becton Dickinson Labware, Bedford, Mass.). The following culture media were tested for ES cells differentiation: DMEM, Williams' medium E (WME), and Iscove's modified Dulbecco's medium (IMDM), all supplemented with 10 to 20% FBS, 0.3 mM monothioglycerol (Sigma-Aldrich), penicillin/streptomycin at standard concentrations, and a range of growth and differentiation factors including hepatocyte growth factor (HGF, 20 ng/ml), nerve growth factor (NGF, 100 ng/ml), epidermal growth factor (EGF, 100 ng/ml), acidic fibroblast growth factor (aFGF, 100 ng/ml), basic fibroblast growth factor (bFGF, 100 ng/ml) (Li, X. et al., J. Cell. Biol. 153:811-822 (2001)), all-trans-retinoic acid (RA, 1 μM) (Makita, T. et al., Genes. Dev. 15:889-901 (2001)), oncostatin M (OnM, 10 ng/ml) (Kamiya, A. et al., EMBO. J. 18:2127-2136 (1999)), bovine insulin (0.126 U/ml), dexamethasone (100 nM) (all from Sigma-Aldrich), and human insulin (0.126 U/ml) (Eli Lilly and Company, Indianapolis, Ind.). Growth and differentiation factors were added to the culture media at final concentrations as indicated above. In addition, EBs were also cultured in medium that was previously used to induce multipotent adult progenitor cells (MAPC) from mouse bone marrow to differentiate into functional hepatocyte-like cells (Schwartz, R. E. et al., J. Clin. Invest. 109:1291-1302 (2002)).

Culture of Human ES Cells

Human ES cell line H1 was obtained from WiCell Research Institute, Madison, Wis. The ES cells were expanded on STO fibroblast feeder layers in DMEM/F 12 containing 20% Knockout serum, 0.5 mM L-glutamine, 100 μM non-essential amino acid solution, 0.1 mM 2-mercaptoethanol, and bFGF (4 ng/ml) (Invitrogen, Carlsbad, Calif.). To prepare feeder layers, mouse STO fibroblasts were cultured until confluent and treated with 10 μg/ml mitomycin C (Sigma-Aldrich) for 4 hours. Mitomycin C-treated mouse STO fibroblasts were then re-seeded at a density of 2.5-3×10⁴/cm² one day before plating ES cells. As illustrated in FIG. 1B, the differentiation of ES cells was initiated by seeding ES cells on non-coated 100-mm tissue culture dishes without a mouse STO fibroblast feeder layer and LIF. These culture conditions stimulated the formation of non-attached embryoid bodies (EBs). Six days later, EBs were placed, and the expression of hepatocyte-specific genes was examined at day 8-43 (FIG. 1B). Human cells were obtained from an NIH-sponsored procurement network of human tissues and organs resource. Human subject exempt protocols were approved by the Human Subject Review Committee of the University of California, Davis.

Isolation and Culture of Mouse Primary Hepatocytes

Mouse primary hepatocytes were prepared by two-step collagenase digestion (Wu, J. et al., J. Biol. Chem. 275:22213-22219 (2000)). Isolated mouse hepatocytes were seeded at a density of 0.75×10⁶ per well in 6-wells plates precoated with collagen type I in WME containing 10% FBS, 10 mM HEPES, 26 mM NaHCO₃, human insulin (0.02 U/ml), and penicillin/streptomycin at standard concentrations, and incubated at 37° C. in a 5% CO₂ atmosphere. Two hours after plating, culture medium was changed to DMEM, WME, or IMDM, supplemented with 10 to 20% FBS, penicillin/streptomycin at standard concentrations, and a range of growth and differentiation factors including dexamethasone (50 nM), and human insulin (0.063 U/ml). In addition, isolated primary hepatocytes were also cultured in MAPC medium, hepatoblasts medium (Rogler, L. E. Am. J. Pathol. 150:591-602 (1997)), or hepatocyte growth medium (Block, G. D. et al., J. Cell. Biol. 132:1133-1149 (1996)). Medium was changed every other day until RNA extraction.

Quantitative Gene Expression Analysis

Total RNA was extracted with RNeasy Mini Kit (Qiagen, Valencia, Calif.). Real time quantitative RT-PCR was employed for the determination of gene expression as described in detail previously by our group (Wege, H. et al., Gastroenterology 124:432-444 (2003)). In brief, one μg RNA was used to generate cDNA after treatment with deoxyribonuclease I (Invitrogen). First-strand synthesis was performed employing the Thermoscript RT-PCR System (Invitrogen) with random hexamers. Relative mouse or human gene expression analysis (ΔΔCT) was performed by real-time quantitative PCR with the ABI Prism 7700 Sequence Detection System and SYBR PCR Green Master Mix or TaqMan PCR Master Mix (Applied Biosystems, Foster City, Calif.). Primer pairs and hybridization probes for mouse or human albumin, prealbumin (transthyretin), alphal-antitrypsin (α1-AT), glucose-6-phosphatase (G6P), cytokeratin 19 (CK19), γ-glutamyl transferase (GGT), and β-actin, were synthesized, and primer concentrations were optimized for specific amplification at 60° C. (Table 1). Expression levels were normalized using mouse β-actin or human glyceraldehyde phosphate dehydrogenase (GAPDH) as an endogenous control. Normalized expression of hepatocyte-specific genes was compared to levels detected in primary adult mouse or human hepatocytes.

Western Blot Analysis of Albumin

Western blot analysis was performed according to a method described previously (Wege, H. et al., Gastroenterology 124:432-444 (2003)). Briefly, proteins from either human or mouse ES cells after various days of culture were extracted in lysing buffer consisting of 150 mM sodium chloride, 1.0% IGEPAL CA-630 (Sigma-Aldrich), 50 mM Tris-HCl (pH 8.0), and Complete Mini protease inhibitor cocktail (Roche Molecular Biochemicals, Indianapolis, Ind.). Extracted proteins were quantitated using DC Protein Assay (Bio-Rad, Hercules, Calif.), separated by 10% polyacrylamide gel electrophoresis under denaturing conditions, and subsequently transferred to polyvinylidene difluoride membranes (Bio-Rad) for immunodetection. Thirty μg of protein extracted from ES cells and 1 μg from control mouse liver tissue were loaded. A primary rabbit antibody against mouse albumin (Cappel, Aurora, Ohio) diluted 1: 1000 and secondary anti-rabbit IgG conjugated with horseradish peroxidase (Santa Cruz Biotechnology, Santa Cruz, Calif.) at a dilution of 1:2000 were used to probe the membrane for albumin. A primary mouse antibody against monoclonal human albumin (Sigma-Aldrich) diluted 1:2500 and secondary anti-mouse IgG antibodies conjugated with horseradish peroxidase (Santa Cruz Biotechnology) at a dilution of 1: 1000 were used to probe the membrane for human albumin. Primary goat anti-mouse and anti-human actin antibodies (Santa Cruz Biotechnology) diluted 1:200 and 1:1000 diluted secondary anti-goat IgG-horesradish peroxidase conjugates (Santa Cruz Biotechnology) were employed to detect actin as a loading control. After adding ECL Western Blotting Detection Reagents (Amersham Pharmacia Biotech, Piscataway, N.J.), signals were detected by autoradiography. A molecular weight standard (Santa Cruz Biotechnology) was loaded as a marker.

Immunocytochemistry for Albumin

Differentiated human or mouse ES cells incubated on collagen type I pre-coated chamber slides with the optimal culture condition were fixed with 1% paraformaldehyde for 10 minutes at room temperature and post-fixed with ethyl alcohol/acetic acid (2:1) for 5 minutes at −20° C. The fixed cells were incubated sequentially overnight with primary monoclonal antibodies against human albumin (for human ES cells) or with rabbit anti-mouse albumin antibody (for mouse ES cells), the same antibodies used for Western blot analysis, and with anti-mouse IgG- or anti-rabbit IgG-fluorescein isothiocyanate conjugates at room temperature for 30 minutes to visualize albumin under a fluorescence microscope. All antibodies used were diluted 1:80.

Urea Synthesis

Urea synthesis was performed according to a method described previously (Wege, H. et al., Gastroenterology 124:432-444 (2003)). Briefly, to evaluate urea synthesis, differentiated mouse or human ES cells in 6-well plates were incubated in 2 ml of serum-free IMDM for 48 hours following multiple washes with phosphate-buffered saline, pH 7.4. Each well contained approximately 20 EBs. Ten μl of serum-free IMDM supernatant from the cultures and 1 ml of Infinity BUN Reagent (Sigma-Aldrich) were mixed. Absorbance at 340 nm was read at 30, 90, 150, and 210 seconds. Urea values were calculated using a standard curve of several urea concentrations, and were normalized to total DNA content. Data from mouse EC cells were compared to primary mouse hepatocytes, which were isolated and cultured as described (Wu, J. et al., J. Biol. Chem. 275:22213-22219 (2000)).

Statistical Analysis

All data were expressed as means±SEM of at least three independent measurements. ANOVA test and Newman-Keuls tests were employed to evaluate differences among groups and for multiple comparisons. A p-value of less than 0.05 was considered statistically significant.

### Example 2

Development of Culture Conditions for Hepatocyte-Specific Differentiation

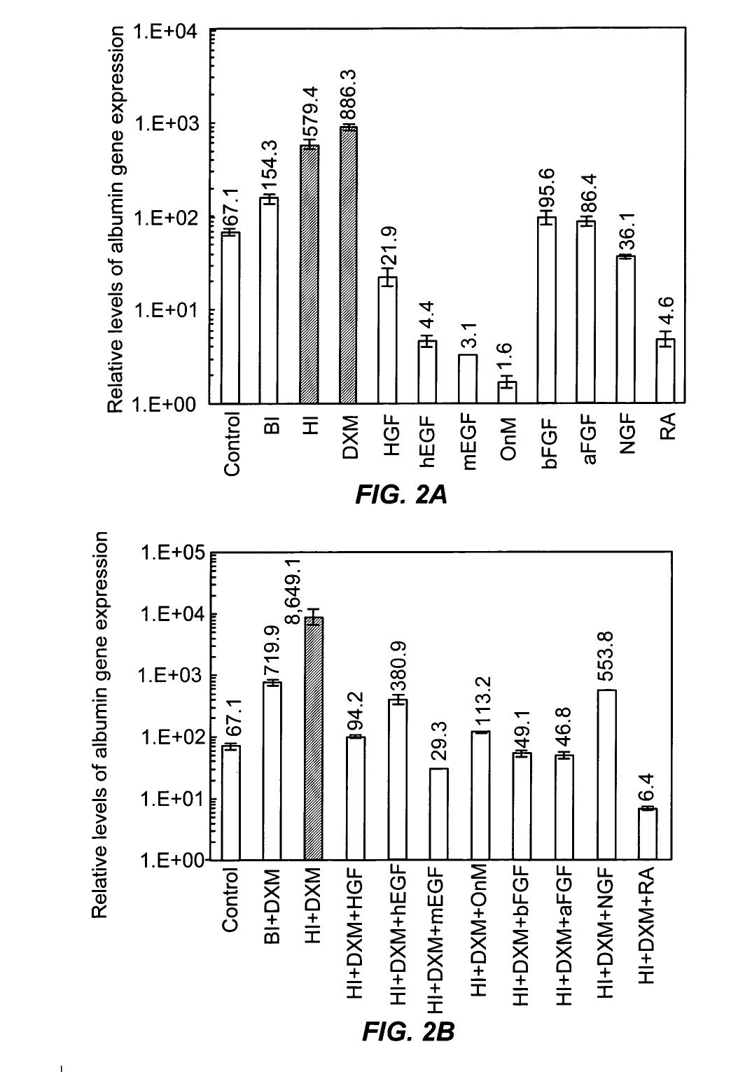

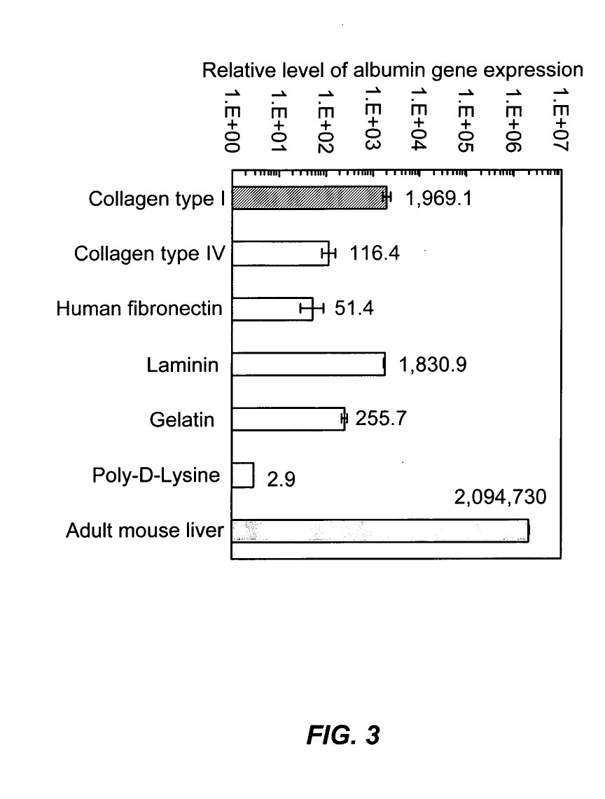

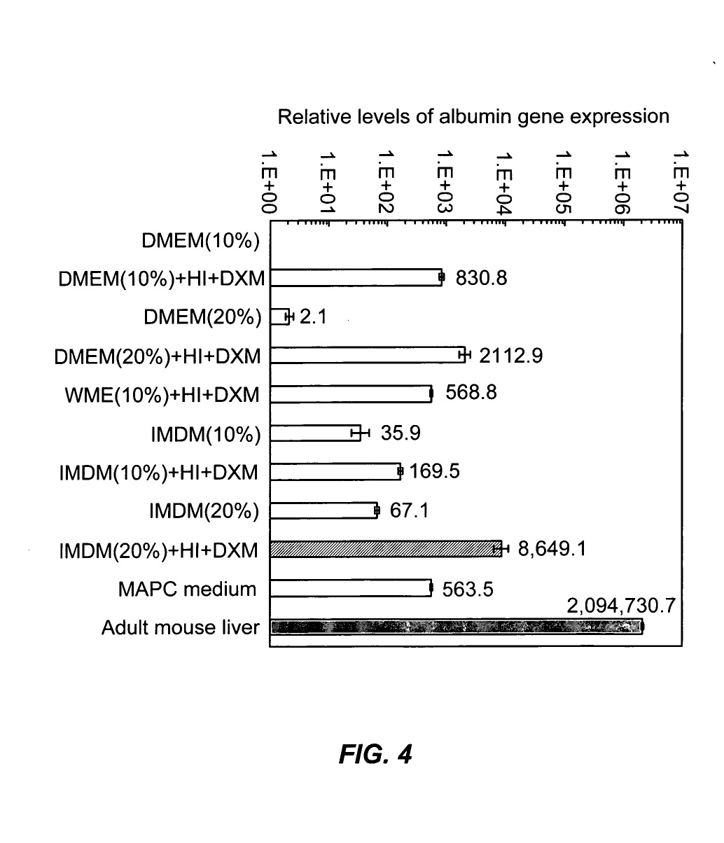

Because albumin synthesis is generally considered to be an excellent marker of hepatocyte differentiation, albumin expression was determined in extensive screening experiments by real-time quantitative RT-PCR to identify the culture condition yielding the highest level of hepatocellular differentiation (albumin expression) in mouse ES cells. In initial experiments, we evaluated the effects of different substratum pre-coatings, culture media, and growth factors. The evaluated growth factors included HGF, NGF, hEGF, mEGF, bFGF, aFGF, RA, OnM, dexamethasone, and human and bovine insulin. Among these growth and differentiation factors, human insulin and dexamethasone were found to be the most effective in enhancing albumin gene expression (FIG. 2A). Furthermore, albumin gene expression was enhanced approximately 10-fold when a combination of human insulin with dexamethasone was added to the culture in comparison to each factor separately (FIG. 2B). On the other hand, other growth and differentiation factors, such as HGF NGF, hEGF, mEGF, bFGF, aFGF, RA, and OnM displayed only minor positive or even negative effects on albumin expression in our culture conditions. These growth and differentiation factors also decreased human insulin and dexamethasone-induced albumin gene expression (FIG. 2B). We also compared six different pre-coatings, including 0.1% gelatin, collagen type I, collagen type IV, laminin, fibronectin and poly-D-lysine. Collagen type I pre-coating resulted in the highest albumin gene expression in ES cells among the substrata tested (FIG. 3). It is evident from FIG. 4 that out of three media tested with either 10% or 20% FBS, IMDM with 20% FBS led to the highest albumin expression in mouse ES cells. Additional experiments indicated that a combination of 0.063 U/ml human insulin and 50 nM dexamethasone was the most potent combination to stimulate albumin expression. Thus, our data indicated that pre-coating culture wells with collagen type I, in combination with IMDM supplemented with 20% FBS, human insulin, and dexamethasone, induced the highest level of albumin expression in mouse ES cells. This culture condition was defmed as the best condition for the differentiation of mouse ES cells along a hepatocyte lineage (subsequently termed “our culture conditions” or the “culture conditions reported here”), and was used in subsequent experiments. Our culture conditions also proved to be more effective in promoting albumin gene expression than conditions recently used to stimulate expression of hepatocyte-specific genes in multipotent adult progenitor cells isolated from mouse bone marrow (FIG. 4).

Time Course of Mouse ES Cell Differentiation and Expression of Hepatocyte-Specific Genes

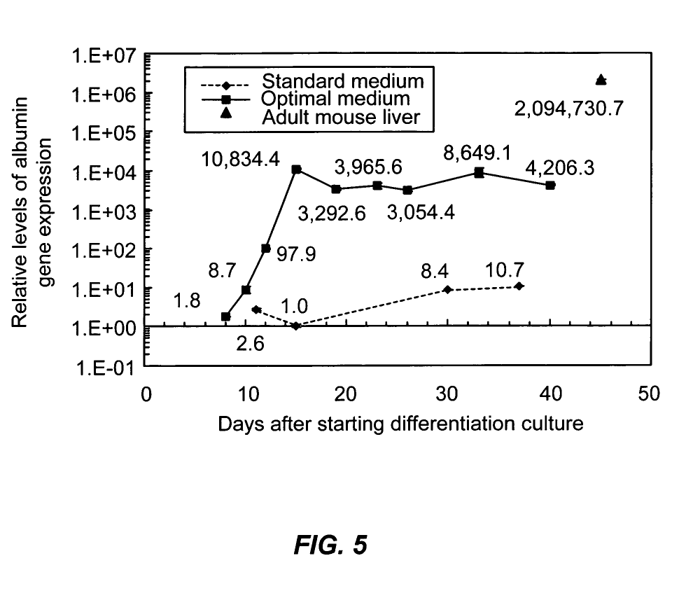

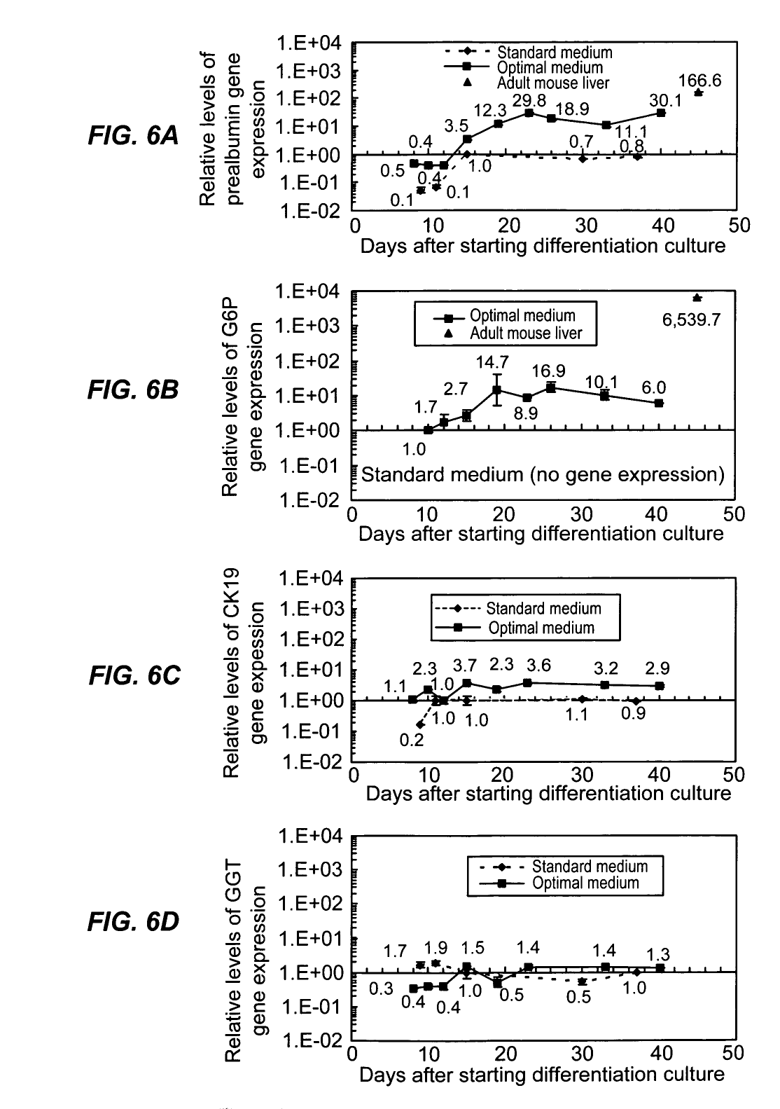

We also examined the time course of albumin gene expression in mouse ES cells cultured in our optimal culture condition. Albumin expression started to increase by day 8, and was sustained at a high level until day 15. The level of albumin mRNA at day 15 was approximately 0.5% of adult mouse liver, and 1000-fold higher than detected in standard culture conditions, including DMEM with 10% FBS (FIG. 5). Furthermore, gene expression of two other hepatocyte-specific genes, prealbumin (also called transthyretin), and G6P was enhanced markedly by the use of the optimal condition. Prealbumin levels in ES cells reached levels approximately 20% of adult mouse liver using the culture condition reported herein, and were far higher than in the standard culture conditions (FIG. 6A). G6P gene expression increased at day 19, and this level of expression was essentially maintained, at approximately 0.25% of the level in adult mouse liver. In contrast to our culture conditions, the standard culture condition did not induce ES cells to express any G6P at all (FIG. 6B). Our culture conditions did not significantly change the expression of two cholangiocyte cell-specific markers, CK19 and GGT (FIGS. 6C, D). In addition, our culture conditions appeared to inhibit the differentiation of ES cells into other cell fates, such as neural cells. The level of glial fibrillary acidic protein gene expression in differentiated ES cells at day 30 cultured under standard condition was approximately 20-fold higher than when using our culture conditions.

Western Blot Analysis and Immunocytochemistry for Albumin in Differentiated Mouse ES Cells

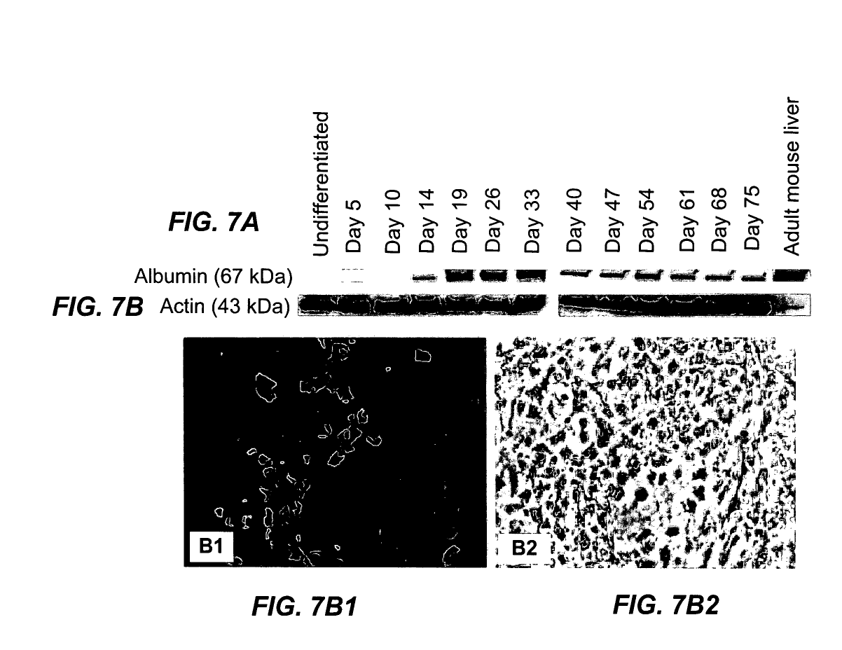

Western blot analysis confirmed the RT-PCR findings. Mouse albumin became positive 14 days after starting the differentiation culture, and peaked at day 19 to a level approximately 7% of adult mouse liver (FIG. 7). Albumin synthesis was maintained thereafter in differentiated ES cells, as demonstrated by Western blot assays, until day 75 duration of the experiment. Immunocytochemistry showed that differentiated ES cells were positive for mouse albumin 26 days after initiating the differentiation culture. This was demonstrated by intense fluorescent signals of albumin staining in the cytosol, and on occasions, positive cells were found in clusters (FIGS. 7A and 7B).

Urea Synthesis in Differentiated Mouse ES Cells

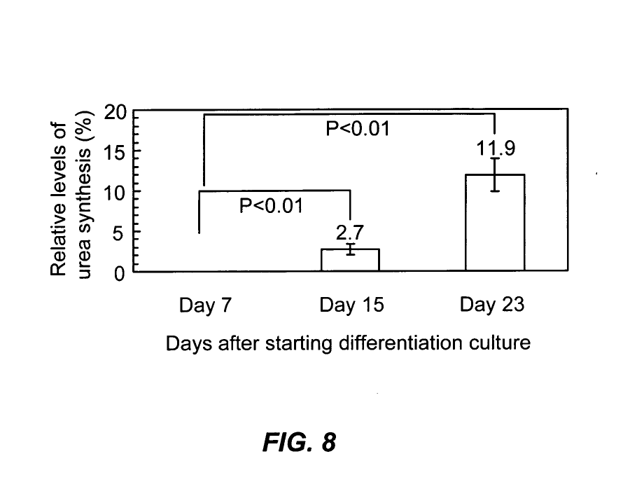

The ability of differentiated ES cells cultured in our culture conditions to synthesize urea was investigated to assess hepatocyte-specific function (FIG. 8). The activity of urea synthesis in ES cells at day 23 (778.7±129.6 μg urea/mg DNA; n=3) was approximately 12% as high as in primary mouse hepatocytes (6950.0±439.8 μg urea/mg DNA; n=3) and significantly higher than at day 7 (p<0.01).

Time Course of Human ES Cell Differentiation and Expression of Hepatocyte-Specific Genes

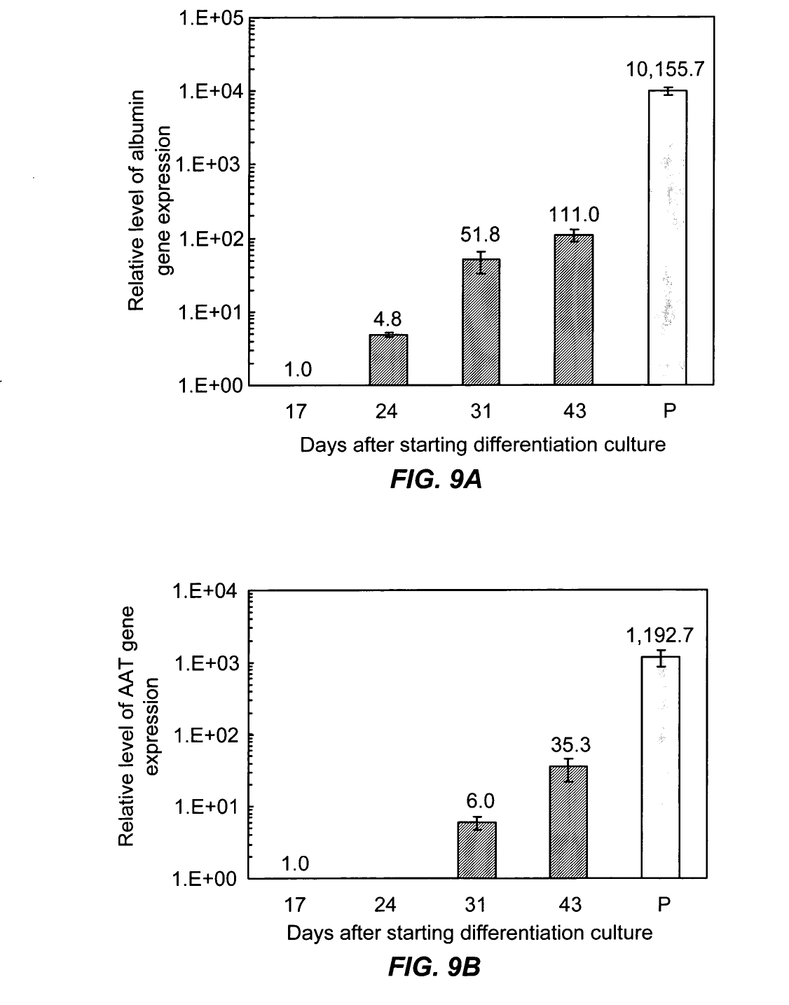

Following the successful differentiation of mouse ES cells along a hepatocyte-like lineage, we examined if the same culture condition would be effective with human ES cells. Albumin expression started to increase by day 17, and was sustained at a high level until day 43. The level of albumin mRNA at day 43 was approximately 1% of adult primary human hepatocytes (FIG. 9A). Furthermore, the gene expression of another hepatocyte-specific gene, human α1-AT was enhanced markedly by the use of our culture conditions, reaching approximately 3% of adult primary human hepatocytes using the culture conditions reported herein (FIG. 9B). Of note, the mRNA levels of cholangiocyte genes, CK-19 and GGT, as well as α-cardiac myosin, were not enhanced during long term culture in the culture conditions reported herein.

Western Blot Analysis and Immunocytochemistry for Albumin in Differentiated Human ES Cells

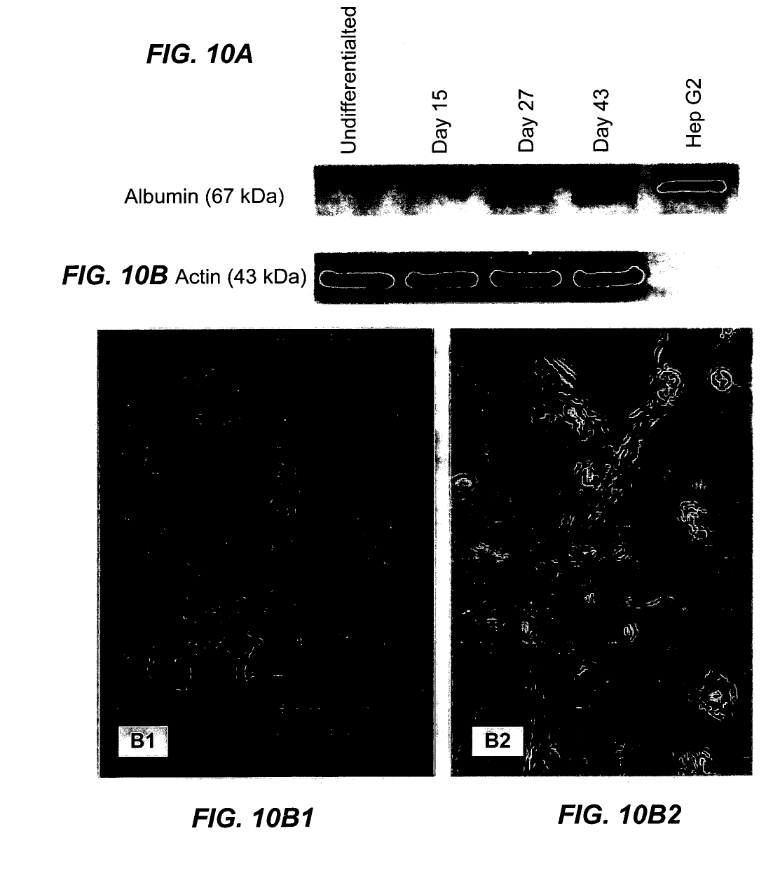

Both Western blot analysis and immunocytochemistry confirmed the RT-PCR findings. Human albumin became positive after 27 days of culture as indicated by Western blot analysis, at a level approximately 1% of that found in a human hepatoma cell line, Hep G2 (FIG. 10A). This cell line is known to synthesize albumin at levels very similar to primary hepatocytes (Wege, H. et al., Gastroenterology 124:432-444 (2003)). Albumin synthesis was maintained thereafter in the differentiated ES cells until at least day 43 (duration of the experiment). Immunocytochemistry for human albumin showed that no positive staining was found in any cells prior to day 17 of differentiating culture. At all later time points tested, differentiated human EC cells were positive for albumin in clumps of cells or as individual cells (FIGS. 10B1 and B2). The staining has been positive to day 54, the length of the immunocytochemical experiment to date.

Urea Synthesis in Differentiated Human ES Cells

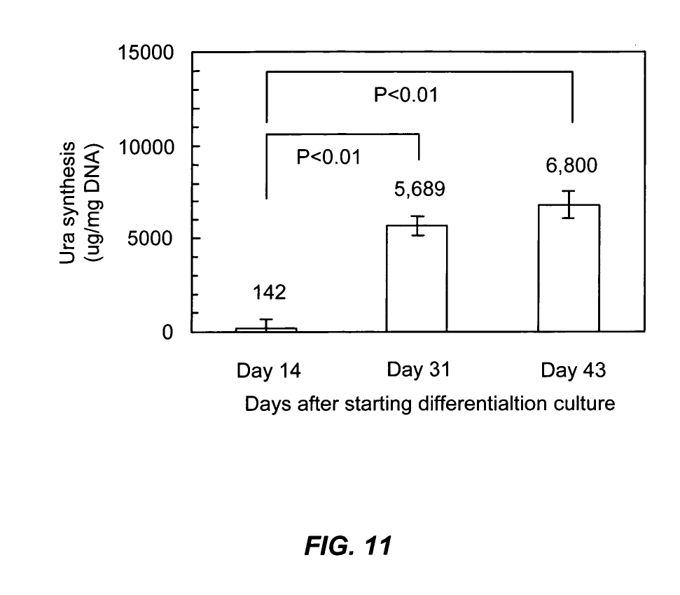

The ability of differentiated human ES cells cultured in our culture conditions to synthesize urea was also investigated to assess hepatocyte-specific function (FIG. 11). The activity of urea synthesis in human ES cells at day 43 (6800.0±529.2±g urea/mg DNA; n=3) was significantly higher than at day 14 (141.7±122.7 μg urea/mg DNA; n=3) (p<0.01). The levels were as high as those produced by cultures of primary rodent hepatocytes.

Culture of Primary Mouse Hepatocytes

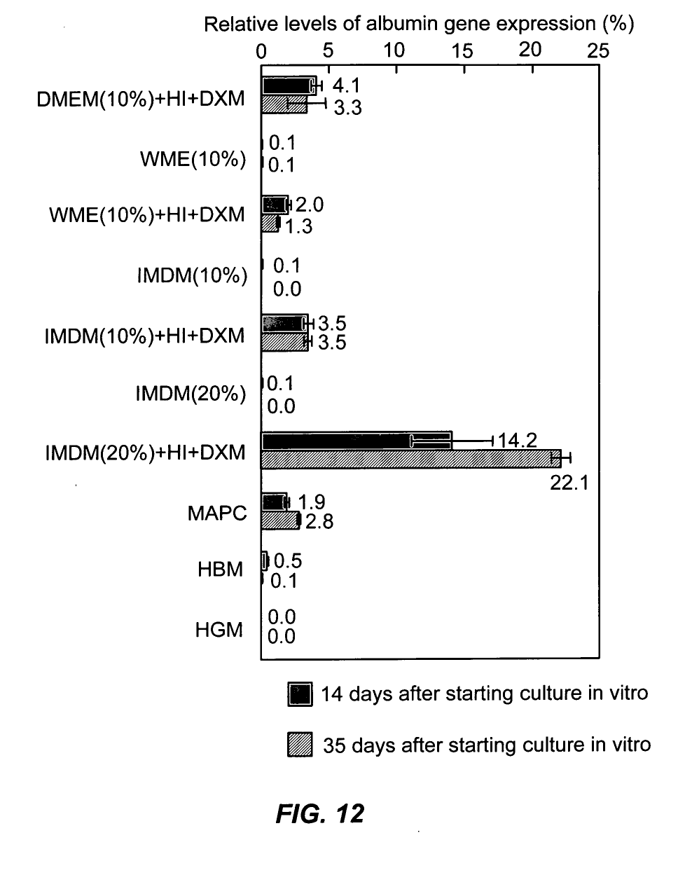

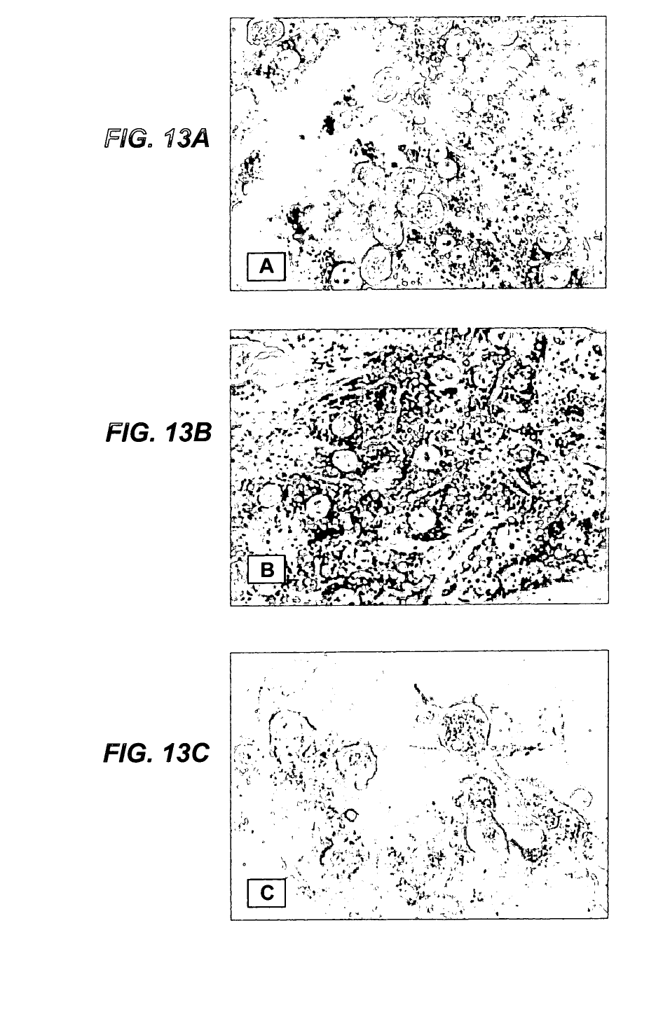

Given the positive results of the culture conditions reported here with both mouse and human ES cells, we tested if this culture condition would be effective in maintaining the phenotype of primary mouse hepatocytes. We examined albumin gene expression in primary mouse hepatocytes cultured in our culture conditions compared to other published hepatocyte differentiation media during long-term culture. The level of albumin mRNA at day 35 (duration of the experiment) was approximately 22% of day one isolated primary mouse hepatocytes, and 10-fold higher than detected in conventional culture conditions (FIG. 12). Furthermore, expression of another hepatocyte-specific gene, prealbumin, was enhanced significantly by the use of the conditions reported when compared to other culture conditions. Prealbumin levels in day 35 primary mouse hepatocytes using the culture conditions reported here reached approximately 5% of isolated mouse primary hepatocytes in the first day after isolation. The levels were considerably higher than with other media. Light microscopy suggested that primary hepatocytes treated for 35 days with the optimal culture condition (FIG. 13B) maintained a morphology similar to primary cells after one day in culture (FIG. 13A), and did not show morphologic evidence of dedifferentiation as did cells cultured under standard conditions (FIG. 13C).

### Example 3

This Example discusses the results of the studies reported conducted in the course of the present invention.

Stem cells are, by definition, capable of self-renewal and differentiation, and thus can theoretically provide a limitless supply of differentiated cells, such as hepatocytes. The findings in the present study demonstrate that mouse ES cells differentiated in vitro into an endodermal cell type with a hepatocyte phenotype and that mouse EBs cultured under the optimal hepatocyte differentiation conditions express significant levels of hepatocyte-specific markers, such as albumin, prealbumin, and G6P, but not the cholangiocyte markers CK 19 and GGT.

To develop culture conditions for hepatocyte differentiation, we tested numerous factors, including HGF, hEGF, mEGF, OnM, aFGF, bFGF, NGF, RA, dexamethasone, and human and bovine insulin. These factors are thought to be important for hepatocyte differentiation during embryonic development, and individual factors or several in combination have been shown to direct the differentiation of ES cells into hepatocyte-like cells, such as aFGF, HGF, OnM, dexamethasone, bovine insulin, transferrin, and selenious acid (Hamazaki, T. et al., FEBS. Lett. 497:15-19 (2001)). However, a recent report showed that the presence or absence of growth and differentiation factors in the presence of serum supplementation did not change albumin production in ES cells (Chinzei, R. et al., Hepatology 36:22-29 (2002)).

In the present study, the combination of human insulin and dexamethasone proved to be very effective in promoting high albumin gene expression in ES cells. However, when other growth and differentiation factors were supplemented, the levels of albumin gene expression were either not affected or were decreased. Thus, our results delineated two beneficial factors, human insulin and dexamethasone, that promoted hepatocyte differentiation of ES cells. In this study, we further demonstrated that the differentiation of ES cells into hepatocyte-like cells could be induced better by placing the cells on collagen type I, than on gelatin, collagen type IV, laminin, fibronectin or poly-D-lysine pre-coated tissue culture plates. Supplementation of IMDM with 20% FBS exhibited much higher levels of albumin expression in ES cells compared to either DMEM or WME. These findings indicated that culture conditions, including medium and substratum pre-coating, as well as FBS and supplementation of growth and differentiation factors, are important factors affecting hepatocyte-specific differentiation of both human and mouse ES cells in vitro. By careful manipulations, a culture condition was defmed to achieve the highest expression level of hepatocyte-specific genes. Moreover, our culture condition had no effect or inhibited differentiation of both human and mouse ES cells into other cell fates, such as cholangiocytes, cardiac muscle cells or neural cells.

Several studies have shown that mouse ES cells can differentiate into albumin-producing cells in vitro (Chinzei, R. et al., Hepatology 36:22-29 (2002); Hamazaki, T. et al., FEBS. Lett. 497:15-19 (2001); Jones, E. A. et al., Exp. Cell. Res. 272:15-22 (2002)). In these studies, the levels of albumin expression in the various culture conditions used were not quantitated, nor compared to normal liver tissue. In this study, we compared albumin and prealbumin mRNA levels in ES cells with levels in adult mouse liver, and the results showed that with the culture conditions developed in this study, albumin expression was approximately 1000-fold higher than when cultured under standard conditions. The level of albumin mRNA reached 0.5% of adult mouse liver, and the albumin protein content in the cells was as high as 7% of adult mouse liver as determined by Western blot analysis. Furthermore, in the hepatocyte differentiation conditions reported here, the activity of urea synthesis at day 23 ES cells was approximately 12% as high as in primary mouse hepatocytes. After we had developed the culture condition to use with mouse ES cells, we then tested whether these culture conditions were beneficial in differentiating human ES cells along a hepatocyte lineage, and in maintaining differentiated function in primary mouse hepatocytes culture. Surprisingly, the same conditions were effective in both experiments. The results were compelling. When tested with the conditions reported here, human ES cells differentiated into albumin-producing cells with albumin mRNA levels at day 43 being approximately 1% as high as in adult human hepatocytes. Significant levels of albumin were also demonstrated in the cells by Western blot analysis. High levels of urea synthesis were also demonstrated in these cells. To our knowledge, this is the first report that differentiated mouse or human ES cells have been shown to express hepatocyte-specific genes at levels even somewhat comparable to fully differentiated hepatocytes. A very recent report represents, to our knowledge, the only other demonstration of human ES cells expressing elements of a hepatocyte phenotype. In that report (Rambhatla, L. et al., Cell Transplant. 12:1-11 (2003)), the differentiated cells were evaluated at only one time point in short term culture (<15 days of differentiation) following treatment with sodium butyrate, which elicited the hepatocyte phenotype but resulted in cell cycle arrest. Our cells, on the other hand, continued to express hepatocyte-specific function for 43 (Western blot) or 54 days (immunocytochemistry) in culture (the length of the experiment), while continuing to proliferate, a characteristic that inhibits liver-specific gene expression. Thus, major differences exist in the effects of the two different culture conditions.

Treatment of primary mouse hepatocytes with our conditions led to levels of albumin mRNA at day 35 of culture (the length of the experiment) that were 22% of newly isolated cells, and a differentiated phenotype was apparent by light microscopy. Because of the tendency of rodent hepatocytes to quickly dedifferentiate when placed in culture, numerous efforts have been made over several decades to better define conditions that maintain their differentiated state during prolonged culture. Our results are at least comparable to previous studies in this arena (Bissell, D. M. et al., J. Clin. Invest. 79:801-812 (1987); Block, G. D. et al., J. Cell. Biol. 132:1133-1149 (1996); Rogler, L. E. Am. J. Pathol. 150:591-602 (1997)), and these results further enforce the notion that our conditions are favorable for the expression of a normal hepatocyte phenotype.

In conclusion, the culture condition described herein induced or maintained a hepatocyte phenotype in several diverse experimental systems. The results provide a basis to 15 establish human hepatocyte-like lines that have utility in cell-based therapeutics, and to maintain primary rodent hepatocytes in a differentiated state in order to employ such stable cultures in toxicology testing, pharmacology testing, and physiology studies.

TABLE 1
Primer pairs and hybridization probes for quantitative PCR

| | | | | | |
|---|---|---|---|---|---|
| | | SEQ ID | | Concen- | Product |
| Gene | Primer-probe sequence 5′-3′ | NO. | 5′-Label | tration | size |

| | | | | | |
|---|---|---|---|---|---|
| Mouse | F: GCAAGGCTGCTGACAAGGA | 1 | | 300 nmol/L | 71 bp |
| albumin | R: GGCGTCTTTGCATCTAGTGACA | 2 | | 300 nmol/L | |
| Mouse | F: TTGCCTCGCTGGACTGGTA | 3 | | 50 nmol/L | 67 bp |
| prealbumin | R: AGGACATTTGGATTCTCCAGCA | 4 | | 300 nmol/L | |
| Mouse G6P | F: TCGTTCCCATTCCGCTTC | 5 | | 300 nmol/L | 71 bp |
| | R: GGCTTCAGAGAGTCAAAGAGATGC | 6 | | 300 nmol/L | |
| Mouse CK19 | F: GTGGCCAGGTCAGTGTGGA | 7 | | 900 nmol/L | 69 bp |
| | R: TCATCTCACTCAGGATCTTGGCTA | 8 | | 900 nmol/L | |
| Mouse GGT | F: CGGTTTGCCTATGCCAAGAG | 9 | | 900 nmol/L | 69 bp |
| | R: GCGGATCACCTGAGACACATC | 10 | | 300 nmol/L | |
| Mouse β- | F: ACGGCCAGGTCATCACTATTG | 11 | | 300 nmol/L | 76 bp |
| actin | R: ATACCCAAGAAGGAAGGCTGGA | 12 | | 50 nmol/L | |
| Human | F: AGTTTGCAGAAGTTTCCAAGTTAGTG | 13 | FAM | 300 nmol/L | 100 bp |
| albumin | T: ACATTCAAGCAGATCTCCATGGCAGCA | 14 | | 300 nmol/L | |
| | R: AGGTCCGCCCTGTCATCAG | 15 | | 50 nmol/L | |
| Human α1- | F: TCGCTACAGCCTTTGCAATG | 16 | FAM | 900 nmol/L | 142 bp |
| AT | T: AGCCTTCATGGATCTGAGCCTCCGG | 17 | | 300 nmol/L | |
| | R: TTGAGGGTACGGAGGAGTTCC | 18 | | 300 nmol/L | |

### Example 4

In addition to the studies reported above, we have added sodium butyrate and dimethyl sulfoxide (DMSO) to the cultures. The levels that were determined to be most effective were 2.5 mM sodium butyrate and 1% DMSO. When they were combined, they further increased the gene expression level of human albumin, alphal-antitrypsin, and transferrin at day 36 of differentiation culture by 19-fold, 2.9-fold and 2.8-fold, respectively, as measured by quantitative RT-PCR.

### Example 5

This Example discusses the construction and use of Self-inactivating Lentivirus Vector (SINLV) expressing a liver-specific marker gene and subsequent FACS analysis

The lentivirus vector built to transduce ESC contains the following components: (1) the essential promoter/enhancer sequences of the LTR were deleted to generate SIN LV, resulting in transcriptional inactivation of the integrated virus. (2) The post-transcriptional regulatory element of the woodchuck hepatitis virus (WPRE) and the central polypurine tract (CPPT) were included because these elements have been shown to enhance lentiviral gene expression in several cell lines, including stem cells. (3) To achieve liver-specific transgene expression, we used the human±1AT promoter as an internal promoter to drive the GFP gene. 293T cells were transfected with the transfer vector construct, packaging construct, Rev expression plasmid, and envelope plasmid coding for G protein of VSV by the calcium phosphate method. Virus was collected over the following 3-4 days and concentrated by ultracentrifugation. The titers of the virus preparations were determined by measuring the amount of HIV-1 p24 gag antigen by ELISA.

Human hepatoma cell lines (Hep G2, Hep 3B, and Huh-7),and non-hepatoma cell lines (prostate cancer cell line, PC-3; colorectal cancer cell line, HCT-116; ovarian cancer cell line, BG1; cervical cancer cell line, Hela cell; kidney cancer cell line, 786-O), were seeded in 6-well plates at 1×10⁵ cells per wall, and were transduced with a lentiviral preparation (30 μL of 3.8×10⁹ transducing units (“TU”)/ml) in total volume of 1 ml growth medium plus Polybrene (8 μg/mL). The cell pellet was used for RNA and DNA isolation. Expression levels were normalized to housekeeping controls, and the expression of GFP in Hep G2 cells was designated as 1, the expression levels of GFP were 1.57 in Huh 7 cells, 2.07 in Hep 3B cells, 0.1 in PC-3 cells, 0.16 in HCT-116 cells, 0.03 in BG1 cells, 0.09 Hela cells, and 0.22 in 786-O cells. These results showed that GFP expression driven by the ±1AT-promoter was significantly higher in hepatoma cells than in non-hepatoma cells. In order to determine whether this difference was caused by transduction efficiency, the relative copy number of HIV p24 from proviral DNA integration into the host genome was also quantified by real-time PCR after transduction.

The p24 copy number in Hep G2 cells was designated as 100. The relative p24 copy numbers were 432 in Huh-7 cells, 249 in Hep 3b cells, 194 in PC-3 cells, 598 in HCT-116 cells, 704 in BG1 cells, 6229 in Hela cells, 127 in 786-O cells. These data demonstrate that high GFP expression in the hepatoma cells was not due to higher transduction efficiency; on the contrary, it showed that transduction with this lentivirus vector containing the GFP marker gene can be employed to demonstrate liver-specific gene expression in transduced cells.

Differentiated hESC were transduced with this lentivirus containing the GFP marker gene at days 32 and 38 after the six-day old embryoid bodies were plated. Undifferentiated human ESC were transduced at day 7. After the transductions, the cells were cultured for at least 12 days, then FACS analysis was performed on a MoFlo Cell Sorter, for detection of GFP-positive cells. Analysis was performed by SUMMIT software (DakoCytomation, Inc.). Forward and side scatter plots were used to exclude dead cells and debris from the histogram analysis plots. The mean fluorescent intensity was determined using cells that had signal intensities higher than the control (non-transduced) cells, which would avoid the intrinsic background fluorescence of the cells. The results showed that the percentage of GFP-positive cells was 1.37% in undifferentiated cells, and 25.1% and 31.19% from two transductions of differentiated cells. These results indicate that the liver-specific lentivirus vector will be effective in enhancing the purity of cells differentiated from ESC and expressing hepatocyte proteins.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A cell differentiated from an embryonic stem (ES) cell along a hepatocyte lineage by culturing said ES cell in a medium with growth factors consisting essentially of insulin and dexamethasone.

2. A cell of claim 1, wherein the insulin is present in said medium in a concentration of from 0.010 U/mL to 1.5 U/mL.

3. A cell of claim 1, wherein the insulin is present in said medium at about 0.050 to about 0.075 U/mL.

4. A cell of claim 1, wherein the insulin in said medium is human insulin.

5. A cell of claim 1, wherein the dexamethasone is present in said medium in a concentration of from 15 nM to 150 nM.

6. A cell of claim 1, wherein the dexamethasone is present in said medium in a concentration of from 40 nM to 60 nM.

7. A cell of claim 1, in which said ES cell is a human ES cell.

8. A cell of claim 1, in which said differentiated cell expresses a hepatocyte-specific protein selected from the group consisting of albumin, pre-albumin, glucose-6-phosphatase, and α1-antitrypsin.

9. A cell of claim 1, wherein said medium further comprises between about 15% to about 30% fetal bovine serum (FBS).

10. A cell of claim 9, wherein said medium comprises 20% FBS.

11. A cell of claim 1, in which the medium is Iscove's modified Dulbecco's medium (IMDM).

12. A cell of claim 1, wherein the ES cell is cultured on an extracellular matrix of collagen type 1.

13. A cell of claim 1, wherein said medium further comprises sodium butyrate.

14. A cell of claim 13, wherein said sodium butyrate is present in a concentration between 0.25 mM and about 10 mM.

15. A cell of claim 1, wherein said medium further comprises dimethyl sulfoxide (“DMSO”).

16. A cell of claim 15, wherein said DMSO is present in a concentration between 0.1% and about 10%.

17. A cell of claim 1, wherein said medium further comprises both sodium butyrate and dimethyl sulfoxide.

18. An isolated hepatocyte maintained in culture by culturing said hepatocyte in a medium with growth factors consisting essentially of insulin and dexamethasone.

19. A hepatocyte of claim 18 in which the medium is Iscove's modified Dulbecco's medium (IMDM).

20. A hepatocyte of claim 18, wherein the insulin is present in said medium in a concentration of from 0.010 U/mL to 1.5 U/mL.

21. A hepatocyte of claim 18, wherein the insulin is present in said medium at about 0.050 to about 0.075 U/mL.

22. A hepatocyte of claim 18, wherein the insulin in said medium is human insulin.

23. A hepatocyte of claim 18, wherein the dexamethasone is present in said medium in a concentration of from 15 nM to 150 nM.

24. A hepatocyte of claim 18, wherein the dexamethasone is present in said medium in a concentration of from 40 nM to 60 nM.

25. A hepatocyte of claim 18, in which said hepatocyte cell is a human hepatocyte cell.

26. A hepatocyte of claim 18, wherein said medium further comprises between about 15% to about 30% fetal bovine serum (FBS).

27. A hepatocyte of claim 18, wherein said medium comprises 20% FBS.

28. A hepatocyte of claim 18, wherein the hepatocyte is cultured on an extracellular matrix of collagen type 1.

29. A hepatocyte of claim 18, wherein said medium further comprises sodium butyrate.

30. A hepatocyte of claim 29, wherein said sodium butyrate is present in a concentration between 0.25 mM and about 10 mM.

31. A hepatocyte of claim 18, wherein said medium further comprises dimethyl sulfoxide (“DMSO”).

32. A hepatocyte of claim 31, wherein said DMSO is present in a concentration between 0.1% and about 10%.

33. A hepatocyte of claim 18, wherein said medium further comprises both sodium butyrate and dimethyl sulfoxide.

34. A method of differentiating embryonic stem (ES) cells along a hepatocyte lineage, said method comprising culturing said ES cell in a medium with growth factors consisting essentially of insulin and dexamethasone.

35. A method of claim 34, in which the medium is Iscove's modified Dulbecco's medium (IMDM).

36. A method of claim 34, wherein the insulin is present in said medium in a concentration of from 0.010 U/mL to 1.5 U/mL.

37. A method of claim 34, wherein the insulin is present in said medium at about 0.050 to about 0.075 U/mL.

38. A method of claim 34, wherein the insulin in said medium is human insulin.

39. A method of claim 34, wherein the dexamethasone is present in said medium in a concentration of from 15 nM to 150 nM.

40. A method of claim 34, wherein the dexamethasone is present in said medium in a concentration of from 40 nM to 60 nM.

41. A method of claim 34, in which said ES cell is a human ES cell.

42. A method of claim 34, in which said differentiated cell expresses a hepatocyte-specific protein selected from the group consisting of albumin, pre-albumin, glucose-6-phosphatase, and α1-antitrypsin.

43. A method of claim 34, wherein said medium further comprises between about 15% to about 30% fetal bovine serum (FBS).

44. A method of claim 34, wherein said medium comprises 20% FBS.

45. A method of claim 34, wherein the cell is cultured on an extracellular matrix of collagen type 1.

46. A method of claim 34, wherein said medium further comprises sodium butyrate.

47. A method of claim 46, wherein said sodium butyrate is present in a concentration between 0.25 mM and about 10 mM.

48. A method of claim 46, wherein said medium further comprises dimethyl sulfoxide (“DMSO”).

49. A method of claim 48, wherein said DMSO is present in a concentration between 0.1% and about 10%.

50. A method of claim 46, wherein said medium further comprises both sodium butyrate and dimethyl sulfoxide.

51. A method of maintaining a hepatocyte in culture for an extended period, said method comprising culturing said hepatocyte cell in a medium in which growth factors consist essentially of insulin and dexamethasone.

52. A method of claim 51, in which the medium is Iscove's modified Dulbecco's medium (IMDM).

53. A method of claim 51, wherein the insulin is present in said medium in a concentration of from 0.010 U/mL to 1.5 U/mL.

54. A method of claim 51, wherein the insulin is present in said medium at about 0.050 to about 0.075 U/mL.

55. A method of claim 51, wherein the insulin in said medium is human insulin.

56. A method of claim 51, wherein the dexamethasone is present in said medium in a concentration of from 15 nM to 150 nM.

57. A method of claim 51, wherein the dexamethasone is present in said medium in a concentration of from 40 nM to 60 nM.

58. A method of claim 51, in which said hepatocyte cell is a human hepatocyte cell.

59. A method of claim 51, wherein said medium further comprises between about 15% to about 30% fetal bovine serum (FBS).

60. A method of claim 51, wherein said medium comprises 20% FBS.

61. A method of claim 51, wherein the hepatocyte is cultured on an extracellular matrix of collagen type 1.

62. A method of claim 51, wherein said medium further comprises sodium butyrate.

63. A method of claim 62, wherein said sodium butyrate is present in a concentration between 0.25 mM and about 10 mM.

64. A method of claim 51, wherein said medium further comprises dimethyl sulfoxide (“DMSO”).

65. A method of claim 64, wherein said DMSO is present in a concentration between 0.1% and about 10%.

66. A method of claim 51, wherein said medium further comprises both sodium butyrate and dimethyl sulfoxide.

67. A method of screening a compound for its effects on a hepatocyte or on a hepatocyte activity, said method comprising
(a) contacting the compound to a cell selected from the group consisting of (i) an embryonic stem (ES) cell differentiated along a hepatocyte lineage by culturing said ES cell with a culture medium containing growth factors, wherein said growth factors consist essentially of insulin and dexamethasone, and (ii) an isolated hepatocyte maintained in culture in a culture medium containing growth factors, wherein said growth factors consist essentially of insulin and dexamethasone;
(b) determining any change to the cells of step (a) contacted with said compound or in an activity of said cells of step (a) contacted with said compound; and
(c) correlating the change of step (b) with the effect of the compound on a cell of step (a) or on an activity of said cell.

68. A method of claim 67, in which the medium is Iscove's modified Dulbecco's medium (IMDM).

69. A method of claim 67, wherein the insulin is present in said medium at about 0.050 to about 0.075 U/mL.

70. A method of claim 67, wherein the insulin in said medium is human insulin.

71. A method of claim 67, wherein the dexamethasone is present in said medium in a concentration of from 40 nM to 60 nM.

72. A method of claim 67, in which said cell in step (a) is selected from the group consisting of a human ES cell and a human hepatocyte cell.

73. A method of claim 67, wherein said medium further comprises 20% FBS.

74. A method of claim 67, wherein the cell of step (a) is cultured on an extracellular matrix of collagen type 1.

75. A method of claim 67, wherein said medium of step (a) further comprises sodium butyrate.

76. A method of claim 75, wherein said sodium butyrate is present in a concentration between 0.25 mM and about 10 mM.

77. A method of claim 67, wherein said medium of step (a) further comprises dimethyl sulfoxide (“DMSO”).

78. A method of claim 77, wherein said DMSO is present in a concentration between 0.1% and about 10%.

79. A method of claim 67, wherein said medium further comprises both sodium butyrate and dimethyl sulfoxide.

80. A cell culture for producing one or more liver proteins, said cell culture selected from the group consisting of (i) an embryonic stem (ES) cell differentiated along a hepatocyte lineage by culturing said ES cell with a culture medium containing growth factors, wherein said growth factors consist essentially of insulin and dexamethasone, (ii) an isolated hepatocyte maintained in culture in a culture medium containing growth factors, wherein said growth factors consist essentially of insulin and dexamethasone, and (iii) a combination of cells of (a) and (b).

81. A cell culture of claim 80, in which the medium is Iscove's modified Dulbecco's medium (IMDM).

82. A cell culture of claim 80, wherein the insulin is present in said medium in a concentration of from 0.010 U/mL to 1.5 U/mL.

83. A cell culture of claim 80, wherein the insulin is present in said medium at about 0.050 to about 0.075 U/mL.

84. A cell culture of claim 80, wherein the insulin in said medium is human insulin.

85. A cell culture of claim 80, wherein the dexamethasone is present in said medium in a concentration of from 15 nM to 150 nM.

86. A cell culture of claim 80, wherein the dexamethasone is present in said medium in a concentration of from 40 nM to 60 nM.

87. A cell culture of claim 80, in which said cell in step (a) is selected from the group consisting of a human ES cell and a human hepatocyte cell.

88. A cell culture of claim 80, wherein said medium further comprises between about 15% to about 30% fetal bovine serum (FBS).

89. A cell culture of claim 80, wherein said medium comprises 20% FBS.

90. A cell culture of claim 80, wherein the cell of step (a) is cultured on an extracellular matrix of collagen type 1.

91. A cell culture of claim 80, wherein said medium of step (a) further comprises sodium butyrate.

92. A cell culture of claim 91, wherein said sodium butyrate is present in a concentration between 0.25 mM and about 10 mM.

93. A cell culture of claim 80, wherein said medium of step (a) further comprises dimethyl sulfoxide (“DMSO”).

94. A cell culture of claim 93, wherein said DMSO is present in a concentration between 0.1% and about 10%.

95. A cell culture of claim 80, wherein said medium of step (a) further comprises both sodium butyrate and dimethyl sulfoxide.

96. A method of producing a liver protein, comprising
(a) providing a cell culture selected from the group consisting of (i) a culture of embryonic stem (ES) cells differentiated along a hepatocyte lineage by culturing said ES cells with a culture medium containing growth factors, wherein said growth factors consist essentially of insulin and dexamethasone, (ii) isolated hepatocytes maintained in culture in a culture medium containing growth factors, wherein said growth factors consist essentially of insulin and dexamethasone, and (iii) a combination of cells of (i) and (ii); and
(b) isolating said liver protein from said culture.

97. A method of claim 96, in which the medium is Iscove's modified Dulbecco's medium (IMDM).

98. A method of claim 96, wherein the insulin is present in said medium at about 0.050 to about 0.075 U/mL.

99. A method of claim 96, wherein the insulin in said medium is human insulin.

100. A method of claim 96, wherein the dexamethasone is present in said medium in a concentration of from 15 nM to 150 nM.

101. A method of claim 96, wherein the dexamethasone is present in said medium in a concentration of from 40 nM to 60 nM.

102. A method of claim 96, in which said cell in step (a) is selected from the group consisting of a human ES cell and a human hepatocyte cell.

103. A method of claim 96, wherein said medium further comprises between about 15% to about 30% fetal bovine serum (FBS).

104. A method of claim 96, wherein said medium comprises 20% FBS.

105. A method of claini 96, wherein the cell of step (a) is cultured on an extracellular matrix of collagen type 1.

106. A method of claim 96, wherein said medium of step (a) further comprises sodium butyrate.

107. A method of claim 96, wherein said medium of step (a) further comprises dimethyl sulfoxide (“DMSO”).

108. A method of claim 96, wherein said medium further comprises both sodium butyrate and dimethyl sulfoxide.

109. A method for identifying cells expressing a hepatocyte-like phenotype, said method comprising transducing a population of cells with a lentiviral vector comprising a gene encoding a marker protein, wherein said gene is operably linked to a promoter for proteins exclusively or preferentially expressed in hepatocytes, and identifying cells expressing the marker protein.

110. A method of claim 109, wherein said marker protein is selected from the group consisting of green fluorescent protein, red fluorescent protein, and an antibiotic.

111. A method of claim 109, further comprising selecting cells expressing said marker protein by fluorescence activated cell sorting.


SEQUENCE LISTING (18 sequences)

SEQ ID NO: 1 - DNA, 19 bp, Artificial Sequence
  Description of Artificial Sequencemouse albumin primer-probe sequence F
gcaaggctgctgacaagga

SEQ ID NO: 2 - DNA, 22 bp, Artificial Sequence
  Description of Artificial Sequencemouse albumin primer-probe sequence R
ggcgtctttgcatctagtgaca

SEQ ID NO: 3 - DNA, 19 bp, Artificial Sequence
  Description of Artificial Sequencemouse prealbumin primer-probe sequence F
ttgcctcgctggactggta

SEQ ID NO: 4 - DNA, 22 bp, Artificial Sequence
  Description of Artificial Sequencemouse prealbumin primer-probe sequence R
aggacatttggattctccagca

SEQ ID NO: 5 - DNA, 18 bp, Artificial Sequence
  Description of Artificial Sequencemouse glucose-6-phosphatase (G6P) primer-probe sequence F
tcgttcccattccgcttc

SEQ ID NO: 6 - DNA, 24 bp, Artificial Sequence
  Description of Artificial Sequencemouse glucose-6-phosphatase (G6P) primer-probe sequence R
ggcttcagagagtcaaagagatgc

SEQ ID NO: 7 - DNA, 19 bp, Artificial Sequence
  Description of Artificial Sequencemouse cytokeratin 19 (CK19) primer-probe sequence F
gtggccaggtcagtgtgga

SEQ ID NO: 8 - DNA, 24 bp, Artificial Sequence
  Description of Artificial Sequencemouse cytokeratin 19 (CK19) primer-probe sequence R
tcatctcactcaggatcttggcta

SEQ ID NO: 9 - DNA, 20 bp, Artificial Sequence
  Description of Artificial Sequencemouse gamma-glutamyl transferase (GGT) primer-probe sequence F
cggtttgcctatgccaagag

SEQ ID NO: 10 - DNA, 21 bp, Artificial Sequence
  Description of Artificial Sequencemouse gamma-glutamyl transferase (GGT) primer-probe sequence R
gcggatcacctgagacacatc

SEQ ID NO: 11 - DNA, 21 bp, Artificial Sequence
  Description of Artificial Sequencemouse beta-actin primer-probe sequence F
acggccaggtcatcactattg

SEQ ID NO: 12 - DNA, 22 bp, Artificial Sequence
  Description of Artificial Sequencemouse beta-actin primer-probe sequence R
atacccaagaaggaaggctgga

SEQ ID NO: 13 - DNA, 26 bp, Artificial Sequence
  Description of Artificial Sequencehuman albumin primer-probe sequence F
ngtttgcagaagtttccaagttagtg

SEQ ID NO: 14 - DNA, 27 bp, Artificial Sequence
  Description of Artificial Sequencehuman albumin primer-probe sequence T
acattcaagcagatctccatggcagca

SEQ ID NO: 15 - DNA, 19 bp, Artificial Sequence
  Description of Artificial Sequencehuman albumin primer-probe sequence R
aggtccgccctgtcatcag

SEQ ID NO: 16 - DNA, 20 bp, Artificial Sequence
  Description of Artificial Sequencehuman alpha1-antitrypsin (alpha1-AT) primer-probe sequence F
ncgctacagcctttgcaatg

SEQ ID NO: 17 - DNA, 25 bp, Artificial Sequence
  Description of Artificial Sequencehuman alpha1-antitrypsin (alpha1-AT) primer-probe sequence T
agccttcatggatctgagcctccgg

SEQ ID NO: 18 - DNA, 21 bp, Artificial Sequence
  Description of Artificial Sequencehuman alpha1-antitrypsin (alpha1-AT) primer-probe sequence R
ttgagggtacggaggagttcc